(19) United States
(12) Patent Application Publication
(10) Pub. No.: US 2024/0382406 A1
(43) Pub. Date: Nov. 21, 2024

(54) PROCESS FOR TREATING KERATINOUS MATERIAL, COMPRISING THE MIXING OF AT LEAST TWO AGENTS (A) AND (B)

(71) Applicant: Henkel AG & Co. KGaA, Duesseldorf (DE)

(72) Inventors: Constanze Kruck, Grevenbroich (DE); Ulrike Schumacher, Düsseldorf (DE); Sandra Hilbig, Bochum (DE); Gabriele Weser, Essen (DE)

(21) Appl. No.: 18/785,831

(22) Filed: Jul. 26, 2024

Related U.S. Application Data
(63) Continuation of application No. PCT/EP2022/084460, filed on Dec. 5, 2022.

(30) Foreign Application Priority Data
Jan. 26, 2022 (DE) 102022200864.2

(51) Int. Cl. A61K 8/898; A61Q 5/10

(52) U.S. Cl. CPC A61K 8/898; A61Q 5/10; A61K 2800/43; A61K 2800/882

(57) ABSTRACT

A method for treating keratinous material, including human hair is provided. The method includes such steps as (1) providing an agent (a), the agent (a) containing: (a1) at least one amino-functionalized silicone polymer, and (2) providing an agent (b), the agent (b) comprising: (b1) at least one alkylene glycol of formula (AG), where x is an integer from 1 to 10000, and (b2) at least one solvent selected 1,2-propylene glycol, propylene carbonate, 1,3-propylene glycol, 1,2-butylene glycol, dipropylene glycol, ethanol, isopropanol, diethylene glycol monoethyl ether, glycerol, phenoxyethanol and benzyl alcohol, and optionally providing an agent (c) that includes at least one pigment. The agents (a) and (b) and optionally (c) can be mixed and applied to keratinous material, such as human keratinous material, allowed to act on the hair, and optionally rinsed form the keratinous material.

### FIELD OF INVENTION

The subject matter of the present invention is a method for dyeing keratinous material, in particular human hair, which comprises the application of two different agents (a) and (b). The agent (a) contains at least one amino-functionalized silicone polymer (a1), and the agent (b) contains at least one alkylene glycol and at least one solvent from a particular group. Before application, an application mixture is prepared by mixing the agents (a) and (b), which mixture is applied to the keratinous material, allowed to act and, optionally, washed off again.

A second subject matter of this application is a multi-component packaging unit (kit-of-parts) for dyeing keratinous material, in particular human hair, comprising the agents (a) and (b), which are packaged separately from one another in two different containers.

### BACKGROUND

Changing the shape and color of keratinous material, in particular human hair, represents an important field of modern cosmetics. To change the hair color, the skilled artisan is familiar with a variety of dyeing systems depending on the dyeing requirements. Oxidation dyes are typically used for permanent, intense dyeing with good fastness properties and good gray coverage. Such dyeing agents contain oxidation dye precursors, what are known as developer components and coupler components, which together form the actual dyes under the influence of oxidizing agents such as, for example, hydrogen peroxide. Oxidation dyes are characterized by very long-lasting color results.

When using direct dyes, dyes which are already formed diffuse out of the coloring agent into the hair fiber. In comparison with oxidative hair dyeing, the colors obtained with direct dyes have a lower durability and a more rapid washing out. Colors with direct dyes usually remain on the hair for a period of between 5 and 20 hair washes.

### SUMMARY OF INVENTION

The use of color pigments for brief changes in color on the hair and/or the skin is known. Color pigments are generally understood to mean insoluble dyeing substances. These are present undissolved in the form of small particles in the dyeing formulation and are only deposited from the outside onto the hair fibers and/or the skin surface. They can therefore generally be removed again without leaving residue by washing a few times with surfactant-containing cleaning agents. Various products of this type by the name of hair mascara are available on the market.

The particular advantage of a hair mascara product is that the dyeing compounds, such as pigments, are deposited on the surface of the keratin fibers only in the form of a film. The nature of the keratin fibers themselves itself is thus not changed when the product is applied, so that the application of a hair mascara product is associated with particularly minimal damage to the hair. If the user wishes to regain their original hair color, the dye can be removed from the keratin fiber quickly, completely and without leaving any residue, without damaging the fibers or changing the original hair color. The development of pigment-based keratin dyes is therefore fully on trend.

In current works, the problem of the low durability of this coloring system has been addressed. In this context, it was found that the wash fastness of the coloring results obtained with pigments could be greatly improved by combining the pigments with certain amino-functionalized silicone polymers. In addition, the choice of particularly well-suited pigments and pigment concentrations on dark hair achieved a brighter color result so that lightening was even possible with this coloring system, which was possible until now only with oxidative hair treatment agents (bleaching or decolorizing agents).

The formation of a uniform film of aminosilicone and pigment on the hair is essential for the application of pigment-based dyeing systems, as irregularities in the film thickness are immediately visually noticeable in an inconsistent color result. Uniform wetting or spreadability of the colorant on the hair is a very important factor in the formation of a correspondingly uniform film. The work leading to this invention has shown that spreadability and wetting of the hair with the dye is still in need of improvement. Accordingly, the color result obtained in the dyeing process was also still non-uniform and could not be regarded as optimal.

The object of the present invention was to provide a hair treatment system which is based on aminosilicones and pigments and which can be spread uniformly on the hair and wets it optimally. The good spreadability should also be ensured, in particular, when the agent is applied to longer hair. In addition, the agent should have sufficiently high storage stability. In addition, intense color results with good fastness properties should be achievable.

Surprisingly, it has now been found that the aforementioned object can be achieved with excellent results if keratinous fibers, in particular hair, have been treated with a ready-to-use agent which has been prepared by mixing at least two agents (a) and (b) prior to application. In this case, the agent (a) comprises at least one amino-functionalized silicone polymer (a1). The agent (b) is a cosmetic carrier formulation and contains at least one alkylene glycol (b1) of a formula (AG) and, in addition, at least one specific organic solvent.

When mixing the agent (a) with a cosmetic carrier formulation (b), it was possible in this way to ensure both a complete transfer of the agent (a) into the agent (b) and also a very uniform application of the agent to the hair. Further agents, for example a third agent (c) with pigments, could also be incorporated very well into the mixture of (a) and (b). When the mixture of (a) and (b) (or the mixture of (a) and (b) and (c)) was used on keratin fibers, the aminosilicone was able to be applied very uniformly to the keratin fibers and formed a homogeneous film of uniform thickness there. The work leading to this invention has shown that this method of mixing (a) and (b) in both dyes and other hair treatment compositions ensures uniform application of the aminosilicone.

### DETAILED DESCRIPTION OF THE INVENTION

A first subject matter of the present invention is a method for treating keratinous material, in particular human hair, comprising the following steps:
(1) providing an agent (a), the agent (a) containing:
(a1) at least one amino-functionalized silicone polymer, and
(2) providing an agent (b), the agent (b) containing:
(b1) at least one alkylene glycol of formula (AG),

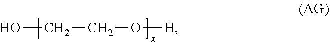

where
x represents an integer from 1 to 10,000, and (b2) at least one solvent from the group consisting of 1,2-propylene glycol, propylene carbonate, 1,3-propylene glycol, 1,2-butylene glycol, dipropylene glycol, ethanol, isopropanol, diethylene glycol monoethyl ether, glycerol, phenoxyethanol and benzyl alcohol, and
(3) optionally providing an agent (c), the agent (c) containing:
(c1) at least one pigment,
(4) preparing an application mixture by mixing agents (a) and (b) and optionally (c),
(5) applying the application mixture prepared in step (4) to the keratinous material,
(6) exposing the keratinous material to the application mixture applied in step (5), and
(7) optionally rinsing out the application mixture.

### Keratinous Material

Keratinous material is understood to mean hair, skin, and nails (such as, for example, fingernails and/or toenails). Furthermore, wool, furs and feathers also fall under the definition of the keratinous material.

Keratinous material is preferably understood to be human hair, human skin and human nails, in particular fingernails and toenails. Keratinous material is very particularly preferably understood to mean keratinous fibers, in particular human hair.

### Agent for Treating Keratinous Material

Agents for treating keratinous material, preferably keratinous fibers, are understood to mean, for example, agents for dyeing the keratin fibers, agents for reshaping or shaping the keratin fibers or also agents for conditioning or caring for the keratin fibers. The method according to the invention shows particularly good suitability in agents for dyeing keratinous fibers, in particular human hair.

Within the scope of this invention, the term “dyeing agent” is used for a coloring of the keratinous material, in particular hair, brought about by use of pigments. In this dyeing process, the pigments are deposited together with the aminosilicone (a1) in a particularly homogeneous and smooth film on the surface of the keratin fibers.

### Agent (a)

In step (1) of the method according to the invention, the agent (a) is provided. For example, agent (a) can be present in a packaging unit or a container and can thereby be made available to the user. The container can be, for example, a sachet, a bottle, a can, a crucible or another container suitable for cosmetic formulations. Agent (a) is characterized by its content of at least one amino-functionalized silicone polymer (a1) and at least one cosmetic oil (a2).

### Amino-Functionalized Silicone Polymer (a1) in Agent (a)

As an ingredient (a1) essential to the invention, the agent (a) contains at least one amino-functionalized silicone polymer. The amino-functionalized silicone polymer can alternatively also be referred to as aminosilicone or amodimethicone.

Silicone polymers are generally macromolecules with a molecular weight of at least 500 g/mol, preferably at least 1000 g/mol, more preferably at least 2500 g/mol and more preferably at least 5000 g/mol which comprise repeating organic units.

The maximum molecular weight of the silicone polymer depends on the degree of polymerization (number of polymerized monomers) and the batch size, and is also determined by the polymerization method. In the context of the present invention, it is preferable if the maximum molecular weight of the silicone polymers is not more than 10⁷ g/mol, preferably not more than 106 g/mol, and particularly preferably not more than 10 g/mol.

The silicone polymers comprise many Si—O repeat units, the Si atoms being able to bear organic residues such as, for example, alkyl groups or substituted alkyl groups. Alternatively, a silicone polymer is therefore also referred to as polydimethylsiloxane.

Corresponding to the high molecular weight of the silicone polymers, these are based on more than 10 Si—O repeat units, preferably more than 50 Si—O repeat units and particularly preferably more than 100 Si—O repeat units, very particularly preferably more than 500 Si—O units.

An amino-functionalized silicone polymer is understood to mean a functionalized silicone which bears at least one structural unit with an amino group. The amino-functionalized silicone polymer preferably bears a plurality of structural units with at least one amino group in each instance. An amino group is understood to mean a primary amino group, a secondary amino group and a tertiary amino group. All these amino groups can be protonated in an acidic environment and are then present in their cationic form.

In principle, it was possible to achieve positive effects with amino-functionalized silicone polymers (a1) when they bore at least one primary, at least one secondary and/or at least one tertiary amino group. However, intense dyeings with the best colorfastness were obtained when an amino-functionalized silicone polymer (a1) containing at least one secondary amino group had been used in the agent (a).

In a very particularly preferred embodiment, a method according to the invention is
characterized in that the agent (a) contains at least one amino-functionalized silicone polymer (a1) having at least one secondary amino group.

The secondary amino groups(s) may be at different positions of the amino-functionalized silicone polymer. Particularly good effects were found when an amino-functionalized silicone polymer (a1) was used that had at least one, preferably multiple, structural units of the formula (Si-amino).

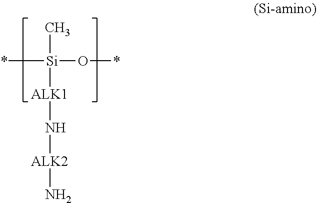

In the structural units of the formula (Si-amino), the abbreviations ALK1 and ALK2 are each independently a linear or branched divalent C₁-C₂₀ alkylene group.

In an additional very particularly preferred embodiment, a method according to the invention is characterized in that the agent (a) contains at least one amino-functionalized silicone polymer (a1) which comprises at least one structural unit of formula (Si-amino),

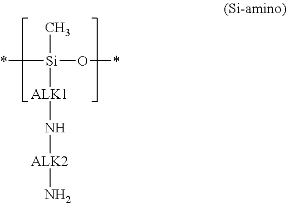

where
ALK1 and ALK2 represent, independently of one another, a linear or branched divalent C₁-C₂₀ alkylene group.

The positions marked with an asterisk (*) in each case indicate the bond to further structural units of the silicone polymer. For example, the silicon atom adjacent to the asterisk can be bonded to an additional oxygen atom, and the oxygen atom adjacent to the asterisk can be bonded to an additional silicon atom or else to a C₁-C₆ alkyl group.

A divalent C₁-C₂₀ alkylene group can alternatively also be termed a double-bond C₁-C₂₀ alkylene group, which means that each moiety ALK1 or ALK2 can have two bonds.

In the case of ALK1, the silicon atom is bonded to the moiety ALK1, and the second bond is between ALK1 and the secondary amino group.

In the case of ALK2, the secondary amino group bonds with the moiety ALK2, and the second bond is formed between ALK2 and the primary amino group.

Examples of a linear divalent C₁-C₂₀-alkylene group are, for example, the methylene group (—CH₂—), the ethylene group (—CH₂—CH₂—), the propylene group (—CH₂—CH₂—CH₂—), and the butylene group (CH₂—CH₂—CH₂—CH₂—). The propylene group (—CH₂—CH₂—CH₂—) is particularly preferred. Starting at a chain length of 3 C atoms, divalent alkylene groups may also be branched. Examples of branched, divalent C₃-C₂₀-alkylene groups are (—CH₂—CH(CH₃)—) and (—CH₂—CH(CH₃)—CH₂—).

In a further particularly preferred embodiment, the structural units of formula (Si-amino) represent repeat units in the amino-functionalized silicone polymer (a1) so that the silicone polymer comprises multiple structural units of formula (Si-amino).

In the following, particularly well suited amino-functionalized silicone polymers (a1) with at least one secondary amino group are listed.

Dyeings with the very best wash fastnesses were obtained when, in the method according to the invention, at least one agent (a) was applied on the keratinous material which contains at least one amino-functionalized silicone polymer (a1) that comprises structural units of formula (Si—I) and formula (Si-II)

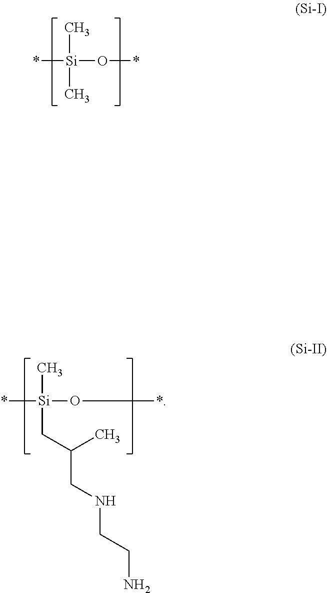

In an additional explicitly very particularly preferred embodiment, a method according to the invention is characterized in that the agent (a) contains at least one amino-functionalized silicone polymer (a1) which comprises structural units of formula (Si—I) and formula (Si-II):

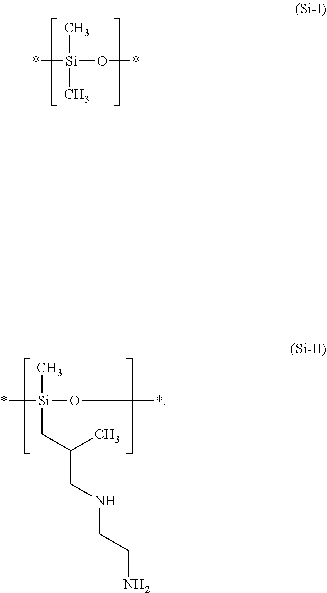

A corresponding amino-functionalized silicone polymer with the structural units (Si—I) and (Si-II) is, for example, the commercial product DC 2-8566 or Dowsil 2-8566 Amino Fluid which is sold commercially by Dow Chemical Company and which bears the designation “Siloxanes and Silicones, 3-[(2-Aminoethyl)amino]-2-methylpropyl Me, Di-Me-Siloxane” and the CAS number 106842-44-8. A further particularly preferred commercial product is Dowsil AP-8568 Amino Fluid, which is likewise commercially sold by Dow Chemical Company.

Within the scope of a further preferred embodiment, a method according to the invention is characterized by the application of an agent (a) to the keratinous material, wherein the agent (a) contains at least one amino-functional silicone polymer (a1) of formula (Si—III),

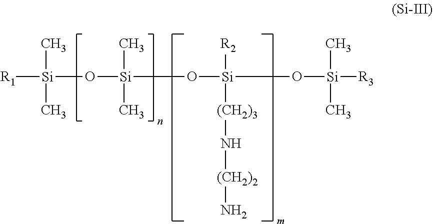

where
   m and n denote numbers that are selected such that the sum (n+m) is in a range of 1 to 1000,
   n is a number in a range of 0 to 999 and m is a number in a range of 1 to 1000,
   R1, R2 and R3, which are identical or different, denote a hydroxyl group or a C1-4 alkoxy group,
   at least one of the R1 to R3 groups denoting a hydroxyl group.

Further methods preferred according to the invention are characterized by the application of an agent (a) to the keratinous material, wherein the agent (a) contains at least amino-functional silicone polymer (a1) of formula (Si—IV),

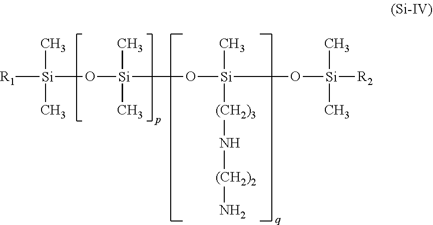

where
   p and q denote numbers that are selected such that the sum (p+q) is in a range of 1 to 1000,
   p is a number in a range of 0 to 999 and q is a number in a range of 1 to 1000, and
   R1 and R2, which are different, denote a hydroxy group or a C1-4 alkoxy group, at least one of the groups R1 to R2 denoting a hydroxy group.

The silicones of the formulas (Si—III) and (Si—IV) differ by the grouping on the Si atom that carries the nitrogen-containing group: In formula (Si—III), R2 denotes a hydroxy group or a C1-4 alkoxy group, whereas the group in formula (Si—IV) is a methyl group. The individual Si moieties, which are labeled with the indices m and n or p and q, need not be present as blocks; instead, the individual units can also be distributed randomly, i.e., in the formulas (Si—III) and (Si—IV), each R1-Si(CH₃)₂ group is not necessarily bound to a —[O—Si(CH₃)₂] moiety.

Methods according to the invention in which an agent (a) containing at least one amino-functional silicone polymer (a1) of formula (Si—V) is applied to the keratin fibers have also proven to be particularly effective with regard to the desired effects

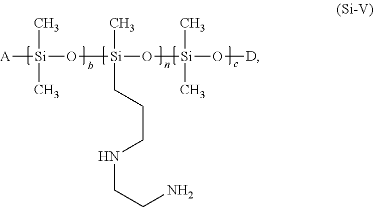

where
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
b, n and c stand for integers between 0 and 1000,
   with the proviso that
   n>0 and b+c>0
   at least one of the conditions A=—OH or D=—H is met.

In the aforementioned formula (Si—V), the individual siloxane units having the indices b, c, and n are randomly distributed, i.e., they are not necessarily block copolymers.

The agent (a) can also comprise one or more different amino-functionalized silicone polymers which are described by the formula (Si—VI):

M(RₐQ_{b}SiO_{(4-a-b)/2)x}(R_{c}SiO_{(4-c)y2)y}M (Si—VI).

In the above formula, R is a hydrocarbon or a hydrocarbon group having 1 to approximately 6 carbon atoms, Q is a polar group of general formula —R¹HZ, in which R¹ is a bivalent linking group bonded to hydrogen and the group Z, composed of carbon and hydrogen atoms, carbon, hydrogen, and oxygen atoms, or carbon, hydrogen and nitrogen atoms, and Z is an organic, aminofunctional group containing at least one aminofunctional group; “a” assumes values in a range of approximately 0 to approximately 2, “b” assumes values in a range of approximately 1 to approximately 3, “a”+“b” is less than or equal to 3, and “c” is a number in a range of approximately 1 to approximately 3, and x is a number in a range of 1 to approximately 2,000, preferably approximately 3 to approximately 50, and most preferably approximately 3 to approximately 25, and y is a number in a range of approximately 20 to approximately 10,000, preferably approximately 125 to approximately 10,000, and most preferably approximately 150 to approximately 1,000, and M is a suitable silicone end group as is known in the prior art, preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl, and the like; alkenyl groups, such as vinyl, halovinyl, alkylvinyl, allyl, haloallyl, and alkylallyl; cycloalkyl groups, such as cyclobutyl, cyclopentyl, cyclohexyl, and the like; phenyl groups; benzyl groups; halohydrocarbon groups such as 3-chloropropyl, 4-bromobutyl, 3,3,3-trifluoropropyl, chlorocyclohexyl, bromophenyl, chlorophenyl, and the like; and sulfur-containing groups, such as mercaptoethyl, mercaptopropyl, mercaptohexyl, mercaptophenyl, and the like; R is preferably an alkyl group containing 1 to approximately 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃C(O)OCH₂CH₂—, —C₆H₄C₆H₄—, —C₆H₄CH₂C₆H₄—; and —(CH₂)₃C(O)SCH₂CH₂—.

Z is an organic, amino-functional group containing at least one functional amino group. A possible formula for Z is NH(CH₂)_{z}NH₂, where z is 1 or more. Another possible formula for Z is —NH(CH₂)_{z}(CH₂)_{zz}NH, in which both z and zz are independently 1 or more, this structure comprising diamino ring structures, such as piperazinyl. Z is most preferably a —NHCH₂CH₂NH₂ group. Another possible formula for Z is N(CH₂)_{z}(CH₂)_{zz}NX₂ or —NX₂, where each X of X₂ is selected independently from the group consisting of hydrogen and alkyl groups having 1 to 12 carbon atoms, and zz is 0.

Q is most preferably a polar, amino-functional group of formula —CH₂CH₂CH₂NHCH₂CH₂NH₂. In the formulas, “a” assumes values in the range of about 0 to about 2, “b” assumes values in the range of about 2 to about 3, “a”+“b” is less than or equal to 3, and “c” is a number in the range of about 1 to about 3. The molar ratio of the RₐQ_{b}SiO_{(4-a-b)/2} units to the R_{c}SiO_{(4-c)/2} units lies within a range of about 1:2 to 1:65, preferably about 1:5 to about 1:65 and most preferably about 1:15 to about 1:20. If one or more silicones of the above formula are used, then the various variable substituents in the above formula can be different in the various silicone components, present in the silicone mixture.

Within the scope of a further preferred embodiment, a method according to the invention is characterized by the application of an agent (a) to the keratinous material, wherein the agent (a) contains at least one amino-functional silicone polymer (a1) of formula (Si—VII),

R′ₐG₃₋ₐ-Si(OSiG₂)ₙ—(OSiG_{b}R′_{2-b})ₘ—O—SiG₃₋ₐ-R′ₐ (Si—VII),
in which:
   G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, or —C(CH₃)₃;
   a represents a number between 0 and 3, in particular 0;
   b represents a number between 0 and 1, in particular 1;
   m and n are numbers whose sum (m+n) is between 1 and 2000, preferably between 50 and 150, n preferably assuming values of 0 to 1999 and in particular from 49 to 149, and m preferably assuming values of 1 to 2000, in particular from 1 to 10;
   R′ is a monovalent group selected from:
   -Q-N(R″)—CH₂—CH₂—N(R″)₂
   -Q-N(R″)₂
   -Q-N⁺(R″)₃A⁻
   -Q-N⁺H(R″)₂A⁻
   -Q-N⁺H₂(R″)A⁻
   -Q-N(R″)—CH₂—CH₂—N⁺R″H₂A⁻,
each Q representing a chemical bond, —CH₂—, —CH₂—CH₂—, —CH₂CH₂CH₂—, —C(CH₃)₂—, —CH₂CH₂CH₂CH₂—, —CH₂C(CH₃)₂—, or —CH(CH₃)CH₂CH₂—,
R″ representing identical or different functional groups from the group —H, -phenyl, -benzyl, —CH₂—CH(CH₃)Ph, from the C₁₋₂₀ alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A representing an anion preferably selected from chloride, bromide, iodide or methosulfate.

Within the scope of a further preferred embodiment, a method according to the invention is characterized by the application of an agent (a) to the keratinous material, wherein the agent (a) contains at least one amino-functional silicone polymer (a1) of formula (Si-VIIa),

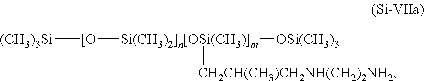

in which m and n are numbers of which the sum (m+n) is between 1 and 2000, preferably between 50 and 150, n preferably assuming values of 0 to 1999 and in particular from 49 to 149 and m preferably assuming values of 1 to 2000, in particular from 1 to 10.

These silicones are designated as trimethylsilylamodimethicones in accordance with the INCI Declaration.

Within the scope of a further preferred embodiment, a method according to the invention is characterized by the application of an agent (a) to the keratinous material, wherein the agent (a) contains at least one amino-functional silicone polymer (a1) of formula (Si-VIIb),

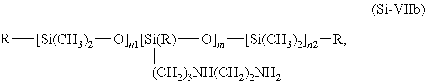

in which R denotes —OH, —O—CH₃, or a —CH₃ group and m, n1 and n2 are numbers whose sum (m+n1+n2) amounts to between 1 and 2000, preferably between 50 and 150, the sum (n1+n2) preferably assuming values from 0 to 1999 and in particular from 49 to 149 and m preferably assuming values from 1 to 2000, in particular from 1 to 10.

According to the INCI Declaration, these amino-functionalized silicone polymers are referred to as amodimethicones.

Irrespective of which amino-functional silicones are used, agents (a) according to the invention that contain an amino-functional silicone whose amine value is above 0.25 meq/g, preferably above 0.3 meq/g, and in particular above 0.4 meq/g are preferred. The amine value here represents the milliequivalents of amine per gram of the amino-functional silicone. Said value can be determined by titration and may also be given in the unit mg KOH/g.

Furthermore, agents (a) which contained a specific 4-morpholinomethyl-substituted silicone polymer (a1) are also suitable for use in the method according to the invention. This amino-functionalized silicone polymer comprises structural units of formulas (Si—VIII) and of the formula (Si—IX):

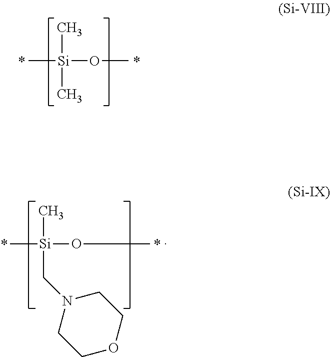

Corresponding 4-morpholinomethyl-substituted silicone polymers are described below.

A very particularly preferred amino-functionalized silicone polymer is known under the name amodimethicone/morpholinomethyl silsesquioxane copolymer and is commercially available in the form of the raw material Belsil ADM 8301 E from Wacker.

For example, a silicone which has structural units of formulas (Si—VIII), (Si—IX) and (Si—X) can be used as 4-morpholinomethyl-substituted silicone:

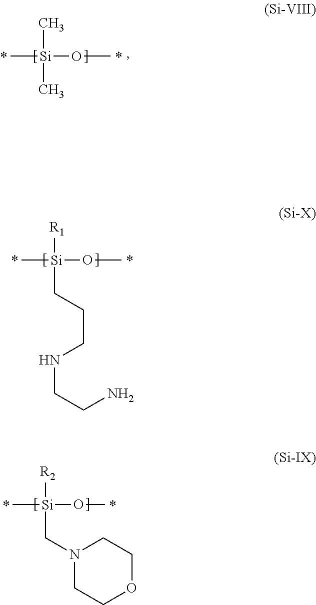

in which
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.

Particularly preferred agents (a) according to the invention contain at least one
4-morpholinomethyl-substituted silicone of the formula (Si—XI):

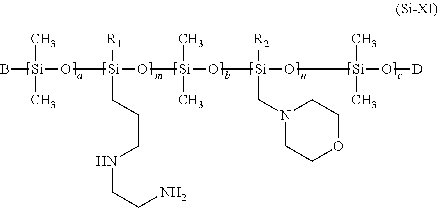

where
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.
B represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group, a, b and c represent, independently of one another, integers between 0 and 1000, with the proviso that a+b+c>0
m and n represent, independently of one another, integers between 1 and 1000, with the proviso that
   at least one of the conditions B═—OH or D=—H is met,
   the units a, b, c, m and n are distributed randomly or in blocks in the molecule.

Structural formula (Si—XI) is intended to indicate that the siloxane groups n and m do not necessarily have to be directly bonded to an end group B or D. Instead, in preferred formulas (Si—VI), a>0 or b>0 and, in particularly preferred formulas (Si—VI), a>0 and c>0; i.e., the terminal group B or D is preferably bonded to a dimethylsiloxy group. In formula (Si—VI) as well, the siloxane units a, b, c, m and n are preferably distributed randomly.

The silicones represented by formula (Si—VI) and used according to the invention can be trimethylsilyl-terminated (D or B═—Si(CH₃)₃), but they may also be dimethylsilylhydroxy-terminated at both ends or dimethylsilylhydroxy- and dimethylsilylmethoxy-terminated at one end. Within the context of the present invention, silicones which are particularly preferably used are selected from silicones in which:
B═—O—Si(CH₃)₂OH and D=—Si(CH₃)₃
B═—O—Si(CH₃)₂OH and D=—Si(CH₃)₂OH
B═—O—Si(CH₃)₂OH and D=—Si(CH₃)₂OCH₃
B═—O—Si(CH₃)₃ and D=—Si(CH₃)₂OH
B═—O—Si(CH₃)₂OCH₃ and D=—Si(CH₃)₂OH.

These silicones lead to enormous improvements in the hair properties of hair treated with the agents according to the invention, and to greatly improved protection during oxidative treatment.

The amino-functionalized silicone polymers (a1) are preferably used in certain amounts in agent (a). Particularly good miscibility and also good properties in terms of application to the hair were obtained if the agent (a) contained, based on the total weight of the agent (a), one or more amino-functionalized silicone polymers (a1) in a total amount of 1 to 100 wt. %, preferably 20 to 100 wt. %, more preferably 30 to 100 wt. %, still more preferably 40 to 100 wt. %, and very particularly preferably 50 to 100 wt. %.

In a further explicitly particularly preferred embodiment, a method according to the invention is characterized in that the agent (a) contains—based on the total weight of the agent (a)—one or more amino-functionalized silicone polymers (a1) in a total amount from 1 to 100 wt. %, preferably 20 to 100 wt. %, more preferably from 30 to 100 wt. %, still more preferably from 40 to 100 wt. %, and very particularly preferably from 50 to 100 wt. %.

At 100% by weight, the agent (a) consists of the aminosilicone and does not contain any other components. This embodiment can be selected, for example, if the agent (a) is to be made available to the user in the form of a concentrate.

However, the agent (a) can also contain one or more further optional components which can be used, for example, to increase the volume of the agent (a) and/or to further improve its rheological properties. In this case, the proportion of the aminosilicone (a1)—based on the total weight of the agent (a)—drops to below 100 wt. % and can also be below 50 wt. % and can, for example, be in the region of 10 to 40 wt. %.

### Liquid Cosmetic Oils (a2) in the Agent (a)

As an optional component (a2), the agent (a) may contain, for example, at least one cosmetic oil that is liquid at 20° C. The oil (a2), which is liquid at 20° C., serves to dilute the aminosilicone (a1), increases the volume of the agent (a) and also improves the flow properties of the agent (a). For the purposes of the present invention, an oil is understood to be an organic liquid which is liquid or flowable at 20° C. and is not miscible with water. An oil is in this case immiscible with water if it has a solubility in water at 20° C. (760 mmHg) of less than 1 wt. %.

The water solubility of the oil can be determined, for example, in the following manner. 1.0 g of the oil is added to a beaker. Water is added up to 100 g. A stirring bar is added, and the mixture is heated to 25° C. on a magnetic stirrer, while stirring. The mixture is stirred for 60 minutes. Thereafter, the aqueous mixture is visually assessed. If a second phase, i.e., a separate oil phase (e.g., in the form of oil droplets) in addition to the water phase, can still be detected in the oil-water mixture after this period, then the solubility of the oil is less than 1 wt. %.

The oil (a2) should improve the miscibility and homogenization of the aminosilico (a1), and therefore it does not itself represent an aminosilicone. For this reason, the oil (a2) is different from the amino-functionalized silicone polymer (a1).

Particularly well-suited oils (a2) can be selected from the group consisting of non-amino-functionalized oligoalkylsiloxanes, non-amino-functionalized silicone polymers, kerosene oils, isoparaffin oils, synthetic C₃-C₁₂ hydrocarbons, di-C₁₂-C₂₃ alkyl ethers, vegetable oils and ester oils.

In a further explicitly very particularly preferred embodiment, a method according to the invention is characterized in that the agent (a) contains at least one cosmetic oil (a2) which is liquid at 20° C. and is selected from the group consisting of non-amino-functionalized oligoalkylsiloxanes, non-amino-functionalized silicone polymers, kerosene oils, isoparaffin oils, synthetic C₃-C₁₂ hydrocarbons, di-C₁₂-C₂₃ alkyl ethers, vegetable oils and ester oils.

Very particularly well suited cosmetic oils (a2) are selected from the group consisting of non-amino-functionalized oligoalkylsiloxanes and non-amino-functionalized silicone polymers.

For the purposes of the invention, oligoalkylsiloxanes are understood to be oligomeric siloxanes which can be linear or cyclic. The oligoalkylsiloxanes are not amino-functionalized, which means that they do not carry any amino group in their structure.

Preferred linear oligoalkylsiloxanes are compounds of the general formula (OAS)

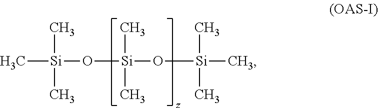

where z represents an integer from 0 to 10. Z preferably represents the numbers 0, 1, 2 or 3.

Very particularly preferred linear oligoalkylsiloxanes are, for example:

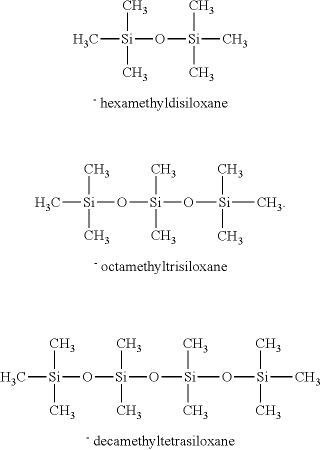

Hexamethyldisiloxane has the CAS number 107-46-0 and can be purchased commercially, for example, from Sigma-Aldrich.

Octamethyltrisiloxane has the CAS number 107-51-7 and is also commercially available from Sigma-Aldrich.

Decamethyltetrasiloxane has the CAS number 141-62-8 and is also commercially available from Sigma-Aldrich.

Preferred cyclic oligoalkylsiloxanes are compounds of the general formula (OAS-II)

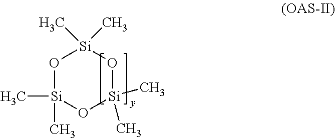

where y represents an integer from 1 to 5. Z preferably represents the numbers 1, 2 or 3.

Very particularly preferred cyclic oligoalkylsiloxanes are, for example, hexamethylcyclotrisiloxane, octamethylcyclotetrasiloxane and decamethylcyclopentasiloxane.

In a further preferred embodiment, a method according to the invention is characterized in that the agent (a) contains at least one cosmetic oil (a2) which is liquid at 20° C. and is selected from the group consisting of oligoalkylsiloxanes of the formula (OAS-I) and/or (OAS-II),

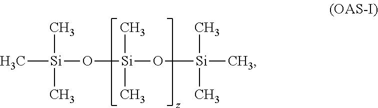

where z stands for an integer from 0 to 10, preferably an integer from 0 to 3 and particularly preferably 0.

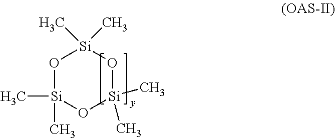

where y represents an integer from 1 to 5, preferably an integer from 1 to 3.

In a further preferred embodiment, a method according to the invention is characterized in that the agent (a) contains at least one cosmetic oil (a2) which is liquid at 20° C. and is selected from the group consisting of hexamethyldisiloxane, octamethyltrisiloxane, decamethyltetrasiloxane, hexamethylcyclotrisiloxane, octamethylcyclotetrasiloxane and/or decamethylcyclopentasiloxane.

The non-amino-functionalized silicone polymers, which are also particularly suitable for solving the problem according to the invention, can alternatively also be referred to as silicone oils. The silicone oils are not amino-functionalized, which means that they do not carry an amino group in their structure.

Silicone oils are polymeric compounds whose molecular weight is at least 500 g/mol, preferably at least 1000 g/mol, more preferably at least 2500 g/mol, and particularly preferably at least 5000 g/mol.

Silicone oils comprise Si—O repeat units, the Si atoms being able to bear organic residues such as, for example, alkyl groups or substituted alkyl groups.

Corresponding to the high molecular weight of the silicone oils, these are based on more than 10 Si—O repeat units, preferably more than 50 Si—O repeat units and particularly preferably more than 100 Si—O repeat units.

In a further preferred embodiment, a method according to the invention is characterized in that the agent (a) contains at least one cosmetic oil (a2) which is liquid at 20° C. and is selected from the group consisting of polydimethylsiloxanes.

It has proven very particularly preferable to use silicone oils in the agent (a), in particular having a viscosity of from 0.5 to 30,000 mm²/s, more preferably from 0.5 to 20,000 mm²/s, still more preferably from 0.5 to 10,000 mm²/s, and particularly preferably from 0.5 to 500 mm²/s, measured according to the ASTM standard D-445.

The ASTM standard D-445 is the standard method for measuring the kinematic viscosity of transparent and opaque fluids.

The viscosity was measured in particular according to ASTM standard D-446, version 06 (D445-06), published June 2006. In this measurement method, the time required for the defined volume of a liquid to flow under defined conditions through the capillary of a calibrated viscometer is measured. With regard to the details of the method, ASMT-D445, refer in particular to ASTM D445-06. Measurement temperature is 25° C. Suitable instruments (such as viscosimeters and thermometers and the corresponding calibrations) are specified in the method.

Within the scope of a further explicitly very particularly preferred embodiment, a method according to the invention is characterized in that the agent (a) contains at least one silicone oil having a viscosity of from 0.5 to 30,000 mm²/s, more preferably from 0.5 to 20,000 mm²/s, still more preferably from 0.5 to 10,000 mm²/s, and particularly preferably from 0.5 to 500 mm²/s, measured according to the ASTM standard D-445 (25° C.).

In principle, various silicone oils can be used in the agent (c), but the use of polydimethylsiloxanes has proven to be particularly advantageous with regard to the improvement of the feel of the hair and the reduction of its oily texture.

For this reason, it is very particularly preferred if agent (a) contains at least one silicone oil (a2) from the group of polydimethylsiloxanes (dimethicones).

The silicone oils from the group of linear polydimethylsiloxanes are compounds of the general structure (PDMS)

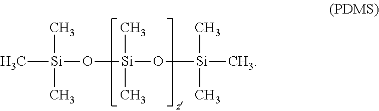

In this case, z′ is selected such that the dimethicones are liquid and preferably have the aforementioned very particularly well suited viscosity ranges.

Preferably, z′ can be an integer from 50 to 100,000, more preferably from 100 to 50,000, particularly preferably from 500 to 50,000.

Corresponding dimethicones are commercially available from various manufacturers. Very particularly well suited is, for example, the dimethicone which is commercially available under the trade name Xiameter PMX 200 Silicone Fluid 50 CS from Dow Chemicals, the viscosity of which is 50 mm²/s (at 25° C.). This dimethicone is most preferred.

Another particularly well suited dimethicone is Xiameter PMX 200 Silicone Fluid 100 CS, also available from Dow Corning, the viscosity of which is 100 mm²/s (measurement at 25° C.).

Another particularly well suited dimethicone is Xiameter PMX 200 Silicone Fluid 350 CS, also available from Dow Corning, the viscosity of which is 350 mm²/s (at 25° C.).

Another particularly well suited dimethicone is Dow Corning 200 fluid 500 cSt, available from Dow Corning, the viscosity of which is 500 mm²/s (at 25° C.).

In another particularly preferred embodiment, a method according to the invention is characterized in that the agent (a) contains at least one cosmetic oil (a2) which is liquid at 20° C., is selected from the group of polydimethylsiloxanes and preferably has a viscosity of 0.5 to 30,000 mm²/s, more preferably from 0.5 to 20,000 mm²/s, still more preferably from 0.5 to 10,000 mm²/s, and particularly preferably from 0.5 to 500 mm²/s, measured according to the ASTM standard D-445 (always measured according to the ASTM standard D-445, 25° C.).

Other well suited oils (a2) are liquid kerosene oils, for example paraffinum liquidum and paraffinum perliquidum, isoparaffin oils, such as isodecane, synthetic hydrocarbons, such as undedane and tridecane, and di-n-alkyl ethers with a total of between 12 and 36 carbon atoms, in particular 12 to 24 C atoms, such as di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether as well as di-tert-butyl ether, di-iso-pentyl ether, di-3-ethyldecyl ether, tert-butyl-n-octyl ether, iso-pentyl-n-octyl ether and 2-methyl-pentyl-n-octyl ether. The compounds 1,3-di-(2-ethyl-hexyl)-cyclohexane (Cetiol® S) and di-n-octyl ether (Cetiol® OE), which are available as commercial products, may be preferred.

Further suitable cosmetic oils (a2) can be selected from the group of vegetable oils. Examples of such oils are sunflower oil, olive oil, soybean oil, rapeseed oil, almond oil, jojoba oil, orange oil, wheat germ oil, peach kernel oil, and the liquid proportions of coconut oil. However, other triglyceride oils, such as the liquid proportions of bovine tallow and synthetic triglyceride oils, are also suitable.

Further suitable cosmetic oils (a2) can also be selected from the group of ester oils. Ester oils are understood to mean the esters of C₆-C₃₀ fatty acids with C₂-C₃₀ fatty alcohols. Preference is given to the monoesters of fatty acids with alcohols having 2 to 24 C atoms. Examples of fatty acid components used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, and technical mixtures thereof. Examples of the fatty alcohol components in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol and brassidyl alcohol and technical mixtures thereof. Particularly preferred according to the invention are isotridecyl isononanoate, neopentyl glycol dihepanoate, isopropyl myristate (Rilanit® IPM), isononanoic acid C16-18 alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V).

Further suitable cosmetic oils (a2) can be selected from the group of dicarboxylic esters, such as di-n-butyl adipate, di-(2-ethylhexyl) adipate, di-(2-ethylhexyl) succinate and di-isotridecyl acyl, and diol esters, such as ethylene glycol dioleate, ethylene glycol-diisotridecanoate, propylene glycol di(2-ethylhexanoate), propylene glycol diisostearate, propylene glycol dipelargonate, butanediol diisostearate, neopentyl glycol dicaprylate, symmetrical, asymmetrical or cyclic esters of carbonic acid with fatty alcohols, glycerol carbonate, and dicaprylyl carbonate (Cetiol® CC).

The cosmetic oils (a2) are preferably contained in certain quantity ranges in agent (a). Very particularly preferably, the agent (a) contains, based on the total weight of the agent (a), one or more cosmetic oils which are liquid at 20° C. in a total amount of from 1 to 99 wt. %, preferably from 30 to 98 wt. %, more preferably from 50 to 97 wt. %, still more preferably from 70 to 96 wt. % and very particularly preferably from 80 to 95 wt. %.

In a further preferred embodiment, a method according to the invention is characterized in that the agent (a) contains, based on the total weight of the agent (a), one or more cosmetic oils which are liquid at 20° C. in a total amount of from 1 to 99 wt. %, preferably from 30 to 98 wt. %, more preferably from 50 to 97 wt. %, still more preferably from 70 to 96 wt. % and very particularly preferably from 80 to 95 wt. %.

In a further preferred embodiment, a method according to the invention is characterized in that the agent (a) contains, based on the total weight of the agent (a), one or more non-amino-functionalized oligoalkylsiloxanes in a total amount of from 1 to 99 wt. %, preferably from 30 to 98 wt. %, more preferably from 50 to 97 wt. %, still more preferably from 70 to 96 wt. % and very particularly preferably from 80 to 95 wt. %.

In a further preferred embodiment, a method according to the invention is characterized in that the agent (a) contains, based on the total weight of the agent (a), one or more silicone oils from the group of dimethicones in a total amount of from 1 to 99 wt. %, preferably from 30 to 98 wt. %, more preferably from 50 to 97 wt. %, still more preferably from 70 to 96 wt. % and very particularly preferably from 80 to 95 wt. %.

### Agent (b)

In step (2) of the method according to the invention, a cosmetic agent (b) is provided. Like the agent (a), the agent (b) can preferably also be present in a packaging unit or a container and can thereby be made available to the user. The container can be for example a sachet, a bottle, a can, a crucible or another container suitable for cosmetic formulations.

The agent (b) is a cosmetic carrier formulation, and is characterized in that this cosmetic carrier comprises an alkylene glycol of the formula (AG),

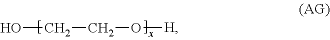

where
x is an integer of from 1 to 10,000.

The alkylene glycols of formula (AG) are protic substances having at least two hydroxyl groups which, due to their repeating —CH2—CH2—O— unit, can also be referred to as polyethylene glycols insofar as x is a value of at least 2. In the alkylene glycols (b1) of the formula (AG), x is an integer from 1 to 10,000. In the context of the work leading to this invention, it was found that these polyethylene glycols exhibit particularly favorable suitability for, on the one hand, improving the fastness properties of the dyeing agents and, on the other hand, also optimally adjusting the viscosity of the agents.

Depending on their chain length, polyethylene glycols are liquid or solid water-soluble polymers. Polyethylene glycols with a molecular mass between 200 g/mol and 400 g/mol are non-volatile liquids at room temperature. PEG 600 has a melting range of 17 to 22° C., and therefore a pasty consistency. With molecular masses above 3000 g/mol, the PEGs are solid substances and are commercially available as flakes or powders.

Especially the use of low molecular weight alkylene glycols (or, respectively, polyethylene glycols) has proven to be well-suited for achieving the object according to the invention. In the event of low molecular weight alkylene glycols (or polyethylene glycols) in the context of the present invention, x denotes an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15.

Within the scope of a further very particularly preferred embodiment, a method according to the invention is characterized in that agent (b) has:
(b1) at least one alkylene glycol of the formula (AG-1), where

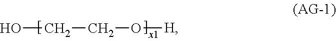

x1 is an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15.

A very particularly preferred low molecular weight polyethylene glycol is PEG-8, for example. PEG-8 comprises, on average, 8 ethylene glycol units (x1=8), has an average molecular weight of 400 g/mol, and bears the CAS number 25322-68-3. PEG-8 is alternatively also referred to as PEG 400 and is commercially available, for example, from APS.

Additional well suited low molecular weight polyethylene glycols are, for example, PEG-6, PEG-7, PEG-9 and PEG-10.

Another well suited polyethylene glycol is PEG-32, for example. PEG-32 comprises 32 ethylene glycol units (x1=32), has a mean molar mass of 1500 g/mol and bears the CAS number 25322-68-3. PEG-32 is alternatively also referred to as PEG 1500 and can, for example, be purchased commercially from Clariant.

Furthermore, the use of high molecular weight polyethylene glycols for achieving the object according to the invention has also proven to be well suited.

High molecular weight polyethylene glycols within the meaning of the present invention can be represented by the formula (AG-2), the index number x2 standing for an integer from 101 to 10,000

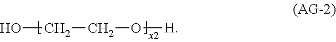

In the case of very well suited high molecular weight polyethylene glycols, x2 denotes an integer from 101 to 1000, preferably an integer from 105 to 800, more preferably an integer from 107 to 600, even more preferably an integer from 109 to 400 and very particularly preferably an integer from 110 to 200.

Within the scope of a further very particularly preferred embodiment, a method according to the invention is characterized in that agent (b) has:
(b1) at least one alkylene glycol of the formula (AG-2), where

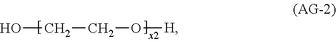

x2 denotes an integer from 101 to 1000, preferably an integer from 105 to 800, more preferably an integer from 107 to 600, even more preferably an integer from 109 to 400, and very particularly preferably an integer from 110 to 200.

A very particularly well suited high molecular weight polyethylene glycol is, for example, PEG 6000, which can be obtained commercially from the National Starch company (China). The molecular weight of PEG 6000 is 6000 to 7500 g/mol, corresponding to an x2 value of 136 to 171.

Another well suited polyethylene glycol is PEG 12000 which, for example, is commercially sold by CG chemicals under the trade name of polyethylene glycol 12000 S (or PEG 12000 S). The molecular weight of PEG 12000 is given at 10,500 to 15,000 g/mol, corresponding to an x2 value of 238 to 341.

Another well suited polyethylene glycol is also PEG 20000 which is commercially available under the trade name polyglycol 20000 P or under the alternative name PEG-350 from Clariant. For PEG 20000, an average molecular weight of 20,000 g/mol is given which corresponds to an x2 value of 454.

Surprisingly, it has been found that dyes which contain both a low molecular weight polyethylene glycol and a high molecular weight polyethylene glycol have particularly favorable application properties, since these agents have both very good fastness properties and are optimized with regard to their rheological profile.

Within the scope of a further very particularly preferred embodiment, a method according to the invention is characterized in that agent (b) has:
(b11) at least one first alkylene glycol of the formula (AG-1), where

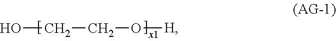

x1 denotes an integer from 1 to 100, preferably an integer from 1 to 80, more preferably an integer from 2 to 60, even more preferably an integer from 3 to 40, even more preferably an integer from 4 to 20, and very particularly preferably an integer from 6 to 15, and
(b12) at least one second alkylene glycol of formula (AG-2), where

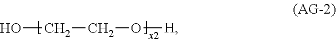

x2 denotes an integer from 101 to 1000, preferably an integer from 105 to 800, more preferably an integer from 107 to 600, even more preferably an integer from 109 to 400, and very particularly preferably an integer from 110 to 200.

To further optimize the application properties, the agent (b) contains the alkylene glycol(s) (b1) of formula (AG), preferably in certain quantity ranges which, for example—based on the total weight of the agent—can be in the range of 10.0 to 99.0 wt. %, preferably 30.0 to 90.0 wt. %, more preferably 40.0 to 80.0 wt. %, and very particularly preferably 45.0 to 60.0 wt. %.

Within the scope of a further particularly preferred embodiment, a method according to the invention is characterized in that the agent (b) contains, based on the total weight of the agent (b), one or more alkylene glycols (b1) of formula (AG) in a total amount of 10.0 to 99.0 wt. %, preferably of 30.0 to 90.0 wt. %, further preferably of 40.0 to 80.0 wt. %, and very particularly preferably of 45.0 to 60.0 wt. %.

Within the scope of a further particularly preferred embodiment, a method according to the invention is characterized in that the agent (b) contains, based on the total weight of the agent (b), one or more alkylene glycols (b1) of formula (AG-1) and/or of formula (AG-2) in a total amount of 10.0 to 99.0 wt. %, preferably of 30.0 to 90.0 wt. %, further preferably of 40.0 to 80.0 wt. %, and very particularly preferably of 45.0 to 60.0 wt. %.

### Water Content in Agent (b)

The agent (b) may also be a water-containing agent that contains water in addition to the alkylene glycol(s). Agent (b) preferably has a low to moderate water content. It has been found that particularly those agents are well-suited which contain, based on the total weight of the agent, 0.1 to 50.0 wt. %, preferably 0.5 to 35.0 wt. %, more preferably 1.0 to 20.0 wt. %, and particularly preferably 1.5 to 15.0 wt. % water.

In another explicit embodiment, a method according to invention is characterized in that the agent (b) contains, based on the total weight of the agent (b), 0.1 to 50.0 wt. %, preferably 0.5 to 35.0 wt. %, more preferably 1.0 to 20.0 wt. %, and particularly preferably 1.5 to 15 wt. % water.

### Solvents (b2) in Agent (b)

The alkylene glycol(s) of formula (AG) contained in agent (b) improve the color result as well as the fastness properties of the dyeings obtained with the mixture of (a) and (b). The touch and texture of the hair can also be optimized by the presence of alkylene glycols (AG). On the other hand, however, tests have shown that the alkylene glycols (AG) can cause problems in terms of the wettability and spreadability of the agent on the hair. In further tests, it has now been found that the wettability and spreadability of the ready-to-use agent on the hair can be massively improved if the agent (b) used in the method additionally contains a solvent (b2) from a specific group in addition to the alkylene glycols (b1). For this reason, the agent (b) contains at least one solvent from the group consisting of 1,2-propylene glycol, propylene carbonate, 1,3-propylene glycol, 1,2-butylene glycol, dipropylene glycol, ethanol, isopropanol, diethylene glycol monoethyl ether, glycerol, phenoxyethanol and benzyl alcohol as the second essential component (b2).

The best results were obtained with 1,2-propylene glycol and/or propylene carbonate. The very best results were obtained with 1,2-propylene glycol.

Alternatively, 1,2-propylene glycol is also referred to as 1,2-propanediol and bears the CAS numbers 57-55-6 [(RS)-1,2-dihydroxypropane], 4254-14-2 [(R)-1,2-dihydroxypropane], and 4254-153 [(S)-1,2-dihydroxypropane]. Ethylene glycol is alternatively also referred to as 1,2-ethanediol and bears the CAS number 107-21-1. Glycerol is alternatively also referred to as 1,2,3-propanetriol and bears the CAS number 56-81-5. Phenoxyethanol has the CAS number 122-99-6.

All the solvents described above are commercially available from various chemicals suppliers such as Aldrich or Fluka.

By using the aforementioned solvents in suitable amounts, particularly good wettability of the keratin fibers or hair can be achieved. It is therefore particularly preferable if the agent (b), based on the total weight of the agent (b), contains one or more solvents (b2) in a total amount of from 1 to 80 wt. %, preferably from 5 to 70 wt. %, more preferably from 10 to 60 wt. %, still more preferably from 15 to 50 wt. % and very particularly preferably from 20 to 40 wt. %.

In a further particularly preferred embodiment, a method according to the invention is characterized in that the agent (b) contains, based on the total weight of the agent (b), one or more solvents in a total amount of from 1 to 80 wt. %, preferably from 5 to 70 wt. %, more preferably from 10 to 60 wt. %, still more preferably from 15 to 50 wt. % and very particularly preferably from 20 to 40 wt. %.

In a further explicitly very particularly preferred embodiment, a method according to the invention is characterized in that the agent (b) contains, based on the total weight of the agent (b), 1 to 80 wt. %, preferably 5 to 70 wt. %, more preferably from 10 to 60 wt. %, still more preferably from 15 to 50 wt. % and very particularly preferably from 20 to 40 wt. % of 1,2-propylene glycol.

### Use of Pigments in the Method for Treating Keratin Fibers

In principle, the method according to the invention is suitable for various methods for treating keratin fibers or hair. These can be all those methods in which a uniform application of the aminosilicone (a1) is of particular importance, such as dyeing, hair straightening or perming. In the case of pigment-based hair dyeing, the non-uniform application of aminosilicone to the hair results in a non-uniform color result and is therefore particularly obvious to the user. For this reason, it is explicitly very particularly preferred if the method according to the invention is a pigment-based dyeing method.

When using agents (a) and (b) in a dyeing method, various embodiments have been found to be particularly well suited. In this embodiment, one or more pigments can be incorporated directly into the agent (b), so that the agent (b) can also be referred to as a dyeing agent.

In a further particularly preferred embodiment, a method according to the invention is characterized in that it is a method for dyeing keratinous material, in particular human hair, and in that the agent (b) contains:
(b3) at least one pigment.

Within the scope of this embodiment, the ready-to-use dye is prepared by mixing agents (a) and (b).

Very particularly preferred (and renumbered accordingly) is thus a method for dyeing keratinous fibers, in particular human hair, comprising the following steps:
(1) providing an agent (a), the agent (a) containing:
(a1) at least one amino-functionalized silicone polymer, and
(2) providing an agent (b), the agent (b) containing:
(b1) at least one alkylene glycol of formula (AG),

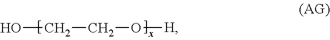

where
x represents an integer from 1 to 10,000, and
(b2) at least one solvent from the group consisting of 1,2-propylene glycol, propylene carbonate, 1,3-propylene glycol, 1,2-butylene glycol, dipropylene glycol, ethanol, isopropanol, diethylene glycol monoethyl ether, glycerol, phenoxyethanol and benzyl alcohol, and
(b3) at least one pigment, and
(3) preparing an application mixture by mixing agents (a) and (b),
(4) applying the application mixture prepared in step (3) to the keratinous material,
(5) exposing the keratinous material to the application mixture applied in step (4), and
(6) optionally rinsing out the application mixture.

Certain pigments, such as, in particular, metal pigments or luster pigments, have proven to be unstable in aqueous solution. Since agent (b), as described above, preferably has a certain proportion of water, sensitive pigments can corrode in this agent. To avoid these incompatibilities, the pigments can therefore also be fabricated in a further separate agent (c).

In a further particularly preferred embodiment, a method according to the invention is characterized in that it is a method for dyeing keratinous material, in particular human hair, and further characterized by the steps of:
(3) providing an agent (c), the agent (c) containing:
(c1) at least one pigment, and
(4) preparing an application mixture by mixing agents (a) and (b) and (c).

Within the scope of this embodiment, the ready-to-use dye is prepared by mixing agents (a) and (b) and (c).

Very particularly preferred is thus a method for dyeing keratinous fibers, in particular human hair, comprising the following steps:
(1) providing an agent (a), the agent (a) containing:
(a1) at least one amino-functionalized silicone polymer, and
(2) providing an agent (b), the agent (b) containing:
(b1) at least one alkylene glycol of formula (AG),

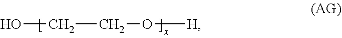

where
x represents an integer from 1 to 10,000, and
(b2) at least one solvent from the group consisting of 1,2-propylene glycol, propylene carbonate, 1,3-propylene glycol, 1,2-butylene glycol, dipropylene glycol, ethanol, isopropanol, diethylene glycol monoethyl ether, glycerol, phenoxyethanol and benzyl alcohol, and
(3) providing an agent (c), the agent (c) containing:
(c1) at least one pigment,
(4) preparing an application mixture by mixing agents (a) and (b) and (c),
(5) applying the application mixture prepared in step (4) to the keratinous material,
(6) exposing the keratinous material to the application mixture applied in step (5), and
(7) optionally rinsing out the application mixture.

When pigment mixtures are used, it is also possible for a first pigment or a first pigment mixture to be used in agent (b) and a second pigment or a second pigment mixture to be used in agent (c).

Pigments within the meaning of the present invention are understood to mean dyeing compounds which have a solubility of less than 0.5 g/L, preferably of less than 0.1 g/L, even more preferably of less than 0.05 g/L, at 25° C. in water. The water solubility can be determined, for example, by means of the method described below: 0.5 g of the pigment is weighed into a beaker. A stir bar is added. Then one liter of distilled water is added. This mixture is heated to 25° C. while stirring with a magnetic stirrer for one hour. If still undissolved components of the pigment are visible in the mixture after this period, the solubility of the pigment is below 0.5 g/L. If the pigment-water mixture cannot be visually assessed due to the high intensity of the pigment that may be finely dispersed, the mixture is filtered. If a portion of undissolved pigments remains on the filter paper, the solubility of the pigment is below 0.5 g/L.

Suitable dye pigments may be of inorganic and/or organic origin.

In a preferred embodiment, an agent (b) according to the invention is characterized in that it contains at least one dyeing compound (b3) from the group of inorganic and/or organic pigments.

Preferred color pigments are selected from synthetic or natural inorganic pigments. Inorganic color pigments of natural origin can be produced, for example, from chalk, ocher, umbra, green soil, burnt sienna or graphite. Furthermore, black pigments such as, for example, iron oxide black, chromatic pigments such as, for example, ultramarine or iron oxide red, and also fluorescent or phosphorescent pigments, can be used as inorganic color pigments.

Colored metal oxides, hydroxides and oxide hydrates, mixed phase pigments, sulfur-containing silicates, silicates, metal sulfides, complex metal cyanides, metal sulfates, chromates and/or molybdates are particularly suitable. Particularly preferred color pigments are black iron oxide (CI77499), yellow iron oxide (CI 77492), red and brown iron oxide (CI 77491), manganese violet (CI 77742), ultramarine (sodium aluminum sulphosilicates, CI 77007, Pigment Blue 29), chromium oxide hydrate (CI77289), Iron Blue (ferric ferrocyanide, C177510) and/or carmine (cochineal).

Color pigments which are likewise particularly preferred according to the invention are colored pearlescent pigments. These are usually based on mica and may be coated with one or more metal oxides. Mica is a phyllosilicate. The most important representatives of these silicates are muscovite, phlogopite, paragonite, biotite, lepidolite, and margarite. In order to produce the pearlescing pigments in conjunction with metal oxides, mica, primarily muscovite or phlogopite, is coated with a metal oxide.

As an alternative to natural mica, synthetic mica coated with one or more metal oxides(s) can also be used as a pearlescent pigment. Particularly preferred pearlescent pigments are based on natural or synthetic mica and are coated with one or more of the aforementioned metal oxides. The color of the respective pigments can be varied by varying the layer thickness of the metal oxide(s).

In a further preferred embodiment, a method according to the invention is characterized in that the agent (b) and/or agent (c) contains at least one pigment from the group of inorganic pigments, which pigment is preferably selected from the group of colored metal oxides, metal hydroxides, metal oxide hydrates, silicates, metal sulfides, complex metal cyanides, metal sulphates, bronze pigments and/or from mica-based colored pigments, which are coated with at least one metal oxide and/or one metal oxychloride.

Examples of particularly suitable color pigments are commercially available, for example, under the trade names Rona®, Colorona®, Xirona®, Dichrona® and Timiron® from the company Merck, Ariabel® and Unipure® from the company Sensient, Prestige® from the company Eckart Cosmetic Colors, and Sunshine® from the company Sunstar.

Very particularly preferred color pigments with the trade name Colorona® are, for example:
Colorona Copper, Merck, MICA, CI 77491 (IRON OXIDES)
Colorona Passion Orange, Merck, Mica, CI 77491 (IRON OXIDES), Alumina
Colorona Patina Silver, Merck, MICA, CI 77499 (IRON OXIDES), CI 77891 (TITANIUM DIOXIDE)
Colorona RY, Merck, CI 77891 (TITANIUM DIOXIDE), MICA, CI 75470 (CARMINE)
Colorona Oriental Beige, Merck, MICA, CI 77891 (TITANIUM DIOXIDE), CI 77491 (IRON OXIDES)
Colorona Dark Blue, Merck, MICA, TITANIUM DIOXIDE, FERRIC FERROCYANIDE
Colorona Chameleon, Merck, CI 77491 (IRON OXIDES), MICA
Colorona Aborigine Amber, Merck, MICA, CI 77499 (IRON OXIDES), CI 77891 (TITANIUM DIOXIDE)
Colorona Blackstar Blue, Merck, CI 77499 (IRON OXIDES), MICA
Colorona Patagonian Purple, Merck, MICA, CI 77491 (IRON OXIDES), CI 77891 (TITANIUM DIOXIDE), CI 77510 (FERRIC FERROCYANIDE)
Colorona Red Brown, Merck, MICA, CI 77491 (IRON OXIDES), CI 77891 (TITANIUM DIOXIDE)
Colorona Russet, Merck, CI 77491 (TITANIUM DIOXIDE), MICA, CI 77891 (IRON OXIDES)
Colorona Imperial Red, Merck, MICA, TITANIUM DIOXIDE (CI 77891), D&C RED NO. 30 (CI 73360)
Colorona Majestic Green, Merck, CI 77891 (TITANIUM DIOXIDE), MICA, CI77288 (CHROMIUM OXIDE GREENS)
Colorona Light Blue, Merck, MICA, TITANIUM DIOXIDE (CI 77891), FERRIC FERROCYANIDE (CI 77510)
Colorona Red Gold, Merck, MICA, CI 77891 (TITANIUM DIOXIDE), CI 77491 (IRON OXIDES)
Colorona Gold Plus MP 25, Merck, MICA, TITANIUM DIOXIDE (CI 77891), IRON OXIDES (CI 77491)
Colorona Carmine Red, Merck, MICA, TITANIUM DIOXIDE, CARMINE
Colorona Blackstar Green, Merck, MICA, CI 77499 (IRON OXIDES)
Colorona Bordeaux, Merck, MICA, CI 77491 (IRON OXIDES)
Colorona Bronze, Merck, MICA, CI 77491 (IRON OXIDES)
Colorona Bronze Fine, Merck, MICA, CI 77491 (IRON OXIDES)
Colorona Fine Gold MP 20, Merck, MICA, CI 77891 (TITANIUM DIOXIDE), CI 77491 (IRON OXIDES)
Colorona Sienna Fine, Merck, CI 77491 (IRON OXIDES), MICA
Colorona Sienna, Merck, MICA, CI 77491 (IRON OXIDES)
Colorona Precious Gold, Merck, Mica, CI 77891 (Titanium dioxide), Silica, CI 77491 (IRON OXIDES), Tin oxide
Colorona Sun Gold Sparkle MP 29, Merck, MICA, TITANIUM DIOXIDE, IRON OXIDES, MICA, CI 77891, CI 77491 (EU)
Colorona Mica Black, Merck, CI 77499 (Iron oxides), Mica, CI 77891 (Titanium dioxide)
Colorona Bright Gold, Merck, Mica, CI 77891 (Titanium dioxide), CI 77491(Iron oxides)
Colorona Blackstar Gold, Merck, MICA, CI 77499 (IRON OXIDES)
Additional particularly preferred color pigments with the trade name Xirona® are, for example:
Xirona Golden Sky, Merck, Silica, CI 77891 (Titanium Dioxide), Tin Oxide
Xirona Caribbean Blue, Merck, Mica, CI 77891 (Titanium Dioxide), Silica, Tin Oxide
Xirona Kiwi Rose, Merck, Silica, CI 77891 (Titanium Dioxide), Tin Oxide
Xirona Magic Mauve, Merck, Silica, CI 77891 (Titanium Dioxide), Tin Oxide.
In addition, particularly preferred color pigments with the trade name Unipure® are, for example:
Unipure Red LC 381 EM, Sensient CI 77491 (Iron Oxides), Silica
Unipure Black LC 989 EM, Sensient, CI 77499 (Iron Oxides), Silica
Unipure Yellow LC 182 EM, Sensient, CI 77492 (Iron Oxides), Silica

The organic pigments according to the invention are correspondingly insoluble organic dyes or color lakes which may be selected, for example, from the group of nitroso, nitro, azo, xanthene, anthraquinone, isoindolinone, isoindoline, quinacridone, perinone, perylene, diketopyrrolopyorrole, indigo, thioindido, dioxazine, and/or triarylmethane compounds.

Particularly well suited organic pigments can for example include carmine, quinacridone, phthalocyanine, sorghum, blue pigments with the Color Index numbers CI 42090, CI 69800, CI 69825, CI 73000, CI 74100 or CI 74160, yellow pigments with the Color Index numbers CI 11680, CI 11710, CI 15985, CI 19140, CI 20040, CI 21100, CI 21108, CI 47000 or CI 47005, green pigments with the Color Index numbers CI 61565, CI 61570 or CI 74260, orange pigments with the Color Index numbers CI 11725, CI 15510, CI 45370 or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In another particularly preferred embodiment, a method according to the invention is characterized in that agent (b) and/or agent (c) contains at least one pigment from the group of organic pigments, which is preferably selected from the group of carmine, quinacridone, phthalocyanine, sorghum, blue pigments with the Color Index numbers CI 42090, CI 69800, CI 69825, CI 73000, CI 74100 or CI 74160, yellow pigments with the Color Index numbers CI 11680, CI 11710, CI 15985, CI 19140, CI 20040, CI 21100, CI 21108, CI 47000 or CI 47005, green pigments with the Color Index numbers CI 61565, CI 61570 or CI 74260, orange pigments with the Color Index numbers CI 11725, CI 15510, CI 45370 or CI 71105, and red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color lake. The term color lake within the meaning of the invention is understood to mean particles which comprise a layer of absorbed dyes, with the unit consisting of particles and dye being insoluble under the above-mentioned conditions. The particles may be, for example, inorganic substrates which may be aluminum, silica, calcium borosilicate, calcium aluminum borosilicate or aluminum.

For example, the alizarin color lake can be used as the color lake.

For the dyeing of the keratin fibers, pigments of a specific shape may also have been used. For example, a pigment based on a lamellar and/or lenticular small substrate plate may be used. Furthermore, the dyeing is also possible based on a small substrate plate which comprises a vacuum metalized pigment.

In a further preferred embodiment, a method according to the invention is characterized in that the agent (b) and/or the agent (c) contains at least one pigment selected from the group of pigments based on a lamellar substrate plate, pigments based on a lenticular substrate plate, and vacuum-metalized pigments.

The small substrate plates of this type have an average thickness of at most 50 nm, preferably less than 30 nm, particularly preferably at most 25 nm, for example at most 20 nm. The average thickness of the small substrate plates is at least 1 nm, preferably at least 2.5 nm, particularly preferably at least 5 nm, for example at least 10 nm. Preferred ranges for the thickness of the small substrate plates are 2.5 to 50 nm, 5 to 50 nm, 10 to 50 nm; 2.5 to 30 nm, 5 to 30 nm, 10 to 30 nm; 2.5 to 25 nm, 5 to 25 nm, 10 to 25 nm, 2.5 to 20 nm, 5 to 20 nm and 10 to 20 nm. Preferably, each substrate plate has as uniform a thickness as possible.

Due to the small thickness of the substrate plates, the pigment has a particularly high covering power.

The substrate plates have a preferably monolithic structure. Monolithic in this context means consisting of a single self-contained unit without fractures, stratifications or inclusions, although structural changes may, however, occur within the substrate plates. The substrate plates are preferably composed homogeneously, i.e., there is no concentration gradient within the plates. In particular, the small substrate plates are not constructed in layers and have no particles distributed therein.

The size of the substrate plate can be matched to the respective application, in particular to the desired effect on the keratinous material. In general, the small substrate plates have an average largest diameter of approximately 2 to 200 μm, in particular approximately 5 to 100 μm.

In a preferred embodiment, the form factor (aspect ratio), expressed by the ratio of the average size to the average thickness, is at least 80, preferably at least 200, more preferably at least 500, particularly preferably more than 750. In this case, the average size of the uncoated small substrate plates is understood to mean the d50 value of the uncoated small substrate plates. Unless stated otherwise, the d50 value was determined using a device of the Sympatec Helos type, having QUIXEL wet dispersion. To prepare the sample, the sample to be investigated was pre-dispersed in isopropanol for a period of 3 minutes.

The small substrate plates may be constructed from any material that can be made into the form of a small plate.

They can be of natural origin, but can also be produced synthetically. Materials from which the small substrate plates can be constructed are, for example, metals and metal alloys, metal oxides, preferably aluminum oxide, inorganic compounds, and minerals such as mica and (semi-) precious stones, as well as plastics materials. Preferably, the small substrate plate are made of metal (alloy)s.

Any metal suitable for metallic luster pigments is suitable as the metal. Such metals are, inter alia, iron and steel, and all air-resistant and water-resistant (semi) metals such as, for example, platinum, zinc, chromium, molybdenum and silicon, as well as alloys thereof such as aluminum bronzes and brass. Preferred metals are aluminum, copper, silver and gold. Preferred small substrate plates are small aluminum plates and small brass plates, small substrate plates made of aluminum being particularly preferred.

Lamellar small substrate plates are characterized by an irregularly structured edge, and are also referred to as “cornflakes” due to their appearance.

Due to their irregular structure, pigments based on lamellar small substrate plates produce a high fraction of scattered light. In addition, the pigments based on lamellar small substrate plates do not completely cover the existing color of a keratinous material and, for example, effects can be achieved analogously to a natural graying.

Lenticular (=lens-shaped) small substrate plates have a substantially regular round edge and are also referred to as “silver dollars” due to their appearance. Due to their regular structure, the fraction of the reflected light predominates in the case of pigments based on lenticular small substrate plates.

Vacuum metalized pigments (VMP) can be obtained, for example, by releasing metals, metal alloys or metal oxides from correspondingly coated films. These are characterized by a particularly small thickness of the small substrate plates in the range from 5 to 50 nm and by a particularly smooth surface having increased reflectivity. Substrate plates which comprise a pigment metalized in a vacuum are also referred to as VMP substrate plates in the context of this application. VMP substrate plates of aluminum can be obtained, for example, by releasing aluminum from metalized films.

The small substrate plates made of metal or metal alloy can be passivated, for example by anodizing (oxide layer) or chromatizing.

Uncoated lamellar, lenticular and/or VPM substrate platelets, in particular those made of metal or metal alloy, reflect the incident light to a high degree and produce a light-dark flop. These have proven to be particularly preferred for use in the coloring agent.

Suitable pigments based on a lamellar substrate platelet include, for example, the pigments of the VISIONAIRE series by Eckart.

Pigments based on a lenticular small substrate plate are available, for example, under the name Alegrace® Gorgeous from the company Schlenk Metallic Pigments GmbH.

Pigments based on a substrate platelet, which comprises a vacuum metalized pigment, are available, for example, under the name Alegrace® Marvelous or Alegrace® Aurous from the company Schlenk Metallic Pigments GmbH.

Owing to their excellent light and temperature resistance, the use of the aforementioned pigments in the method according to the invention is very particularly preferred. It is further preferred if the pigments used have a certain particle size. It is therefore advantageous according to the invention if the at least one pigment has a mean particle size D₅₀ from 1.0 to 50 μm, preferably from 5.0 to 45 μm, preferably from 10 to 40 μm, in particular from 14 to 30 μm. The mean particle size D₅₀ can be determined, for example, using dynamic light scattering (DLS).

In a further very particularly preferred embodiment, a method according to the invention is characterized in that the agent (b) contains, based on the total weight of the respective agent, one or more pigments (b3) in a total amount of 0.05 to 10.0 wt. %, preferably 0.1 to 7.0 wt. %, more preferably 0.2 to 5.0 wt. % and very particularly preferably 0.3 to 3.0 wt. %.

In a further very particularly preferred embodiment, a method according to the invention is characterized in that the agent (c) contains, based on the total weight of the respective agent, one or more pigments (c1) in a total amount of 0.05 to 100.0 wt. %, preferably 3 to 95 wt. %, more preferably 6 to 90 wt. % and very particularly preferably 8 to 80 wt. %.

Addition Products of C₁-C₆ Alkylene Oxide(s) to the Esters of C₁₂-C₃₀ Fatty Acids and Aromatic C₁-C₁₂ Alcohols (b4)

To further improve the fastness properties, such as rub fastness and wash fastness, the agents according to the invention may additionally contain, as an optional component, at least one addition product of C₁-C₆ alkylene oxide(s) to the esters of C₁₂-C₃₀ fatty acids and aromatic C₁-C₁₂ alcohols, which fall under the compounds of general formula (AFE-I)

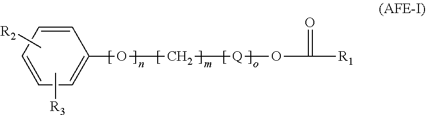

where
R1 represents a saturated or unsaturated C₁₁-C₂₉ alkyl group,
R1, R3 represent, independently of one another, a hydrogen atom, a C₁-C₆ alkyl group, a hydroxy group, or a C₁-C₆ alkoxy group.
n represents the number 0 or 1,
m represents an integer from 0 to 6,
represents an integer from 1 to 60, and
Q represents a structural unit —O—CH₂—CH₂—, —O—CH(CH₃)—CH₂— or —O—CH₂—CH(CH₃)—
with the proviso that if m is equal to 0, n is also equal to 0.

Within the scope of another preferred embodiment, a method according to the invention is characterized in that the agent (b) contains:
(b4) at least one alkoxylated fatty acid ester of general formula (AFE-I)

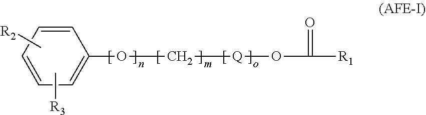

where
R1 represents a saturated or unsaturated C₁₁-C₂₉ alkyl group,
R1, R3 represent, independently of one another, a hydrogen atom, a C₁-C₆ alkyl group, a hydroxy group, or a C₁-C₆ alkoxy group.
n represents the number 0 or 1,
m represents an integer from 0 to 6,
represents an integer from 1 to 60, and
Q represents a structural unit —O—CH₂—CH₂—, —O—CH(CH₃)—CH₂— or —O—CH₂—CH(CH₃)—
with the proviso that if m is equal to 0, n is also equal to 0.

The residue R1 represents a saturated or unsaturated C₁₁C₂₉ alkyl group. An unsaturated C₁₁C₂₃ alkyl group can comprise one or more double bonds and is alternatively also as termed an unsaturated C₁₁C₂₃ alkenyl group. The saturated or unsaturated C₁₁-C₂₉ alkyl group can be linear or branched.

Preferably, R1 represents a linear, saturated or unsaturated C₁-C₂₃ alkyl group.

The residues R2 and R3 are each a hydrogen atom, a C₁C₆ alkyl group, a hydroxy group or a C₁C₆ alkoxy group. Very particularly preferably, the residues R1 and R2 both stand for a hydrogen atom.

The index number n is the number 0 or 1. Preferably, n is the number 0.

The index number m is an integer from 0 to 6. Preferably, m is the number 1.

The index number o is an integer from 1 to 60. Preferably, o is an integer from 1 to 30, more preferably 1 to 20, even more preferably 1 to 10, and very particularly preferably 1 to 5.

The Q residue is a structural unit —O—CH₂—CH₂—, —O—CH(CH₃)—CH₂— or —O—CH₂—CH(CH₃)—. Particularly preferably, Q is a structural unit —O—CH(CH₃)—CH₂— or —O—CH₂—CH(CH₃)—.

If o is a number greater than 1, a plurality of structural units Q are present in the compounds of formula (AFE-I) (or also of formula (AFE-II), in which case each structural unit Q can be selected independently of the other structural units Q.

Within the scope of a further very particularly preferred embodiment, a method according to the invention is characterized in that agent (b) has:
(b4) at least one alkoxylated fatty acid ester of the general formula (AFE-I)

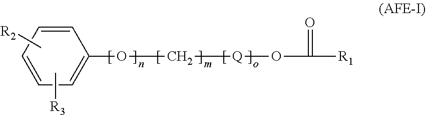

where
R7 represents a saturated or unsaturated C₁₁-C₂₉ alkyl group,
R2 and R4 are both are a hydrogen atom,
n is the number 0,
m is the number 1,
is an integer 1 to 30, preferably 1 to 20, more preferably 1 to 10, and very especially preferably 1 to 5, and
Q is a structural unit —O—CH(CH₃)—CH₂— or —O—CH₂—CH(CH₃)—.

An explicitly very particularly well suited compound of this type is PPG-3 benzyl ether myristate, which is alternatively also referred to as α-(1-oxotetradecyl)-ω-(phenylmethoxy) poly [oxy(methyl-1,2-ethanediyl)] and bears the CAS number 642443-86-5.

PPG-3 benzyl ether myristate can be purchased commercially, for example, under the trade name of Crodamol STS from Croda.

Within the scope of a further, very particularly preferred embodiment, a method according to the invention is characterized in that the agent (b) contains PPG-3 benzyl ether myristate.

The alkoxylated fatty acid esters (b4) are particularly preferably used within specific quantity ranges in the agent according to the invention. Particularly good results were obtained when the agent (b) contained, based on the total weight of the agent (b), one or more alkoxylated fatty acid esters (b4) in a total amount of 0.1 to 20.0 wt. %, preferably 0.5 to 15.0 wt. %, more preferably 1.0 to 10.0 wt. %, still more preferably 1.0 to 8.0 wt. % and very particularly preferably 1.0 to 5.0 wt. %.

Addition Products of C₁-C₆ Alkylene Oxide(s) to Aliphatic C₁-C₂₄ Alkanols (b5)

To further improve the fastness properties, such as rub fastness and wash fastness, the agent (b) according to the invention may additionally contain, as an optional component, at least one addition product of C₁-C₆ alkylene oxides to C1-C24 alkanols.

Within the scope of a further very particularly preferred embodiment, a method according to the invention is further characterized in that agent (b) has:
(b5) at least one addition product of C₁-C₆ alkylene oxide(s) to aliphatic C₁-C₂₄ alkanols.

C₁-C₆ alkylene oxides suitable according to the invention and their preferred and particularly preferred representatives have already been defined in the preceding sections.

The C₁-C₂₄ alkanols are, in accordance with the invention, compounds having 1 to 14 carbon atoms and a hydroxyl group. The following can be mentioned by way of example: methanol, ethanol, 1-propanol, 2-propanol, 1-butanol, 2-butanol, 1-pentanol, 2-pentanol, 3-pentanol, 1-hexanol, 2-hexanol, 3-hexanol, 1-heptanol, 2-heptanol, 3-heptanol, 4-heptanol, 1-octanol, 2-octanol, 3-octanol, 4-octanol, 1-nonanol, 2-nonanol, 3-nonanol, 4-nonanol, 5-nonanol, 1-decanol, 2-decanol, 3-decanol, 4-decanol, 5-decanol, 1-undecanol, 2-undecanol, 3-undecanol, 4-undecanol, 5-undecanol, 6-undecanol, 1-dodecanol, 2-dodecanol, 3-dodecanol, 4-dodecanol, 5-dodecanol, 6-dodecanol, 1-tridecanol, 2-tridecanol, 3-tridecanol, 4-tridecanol, 5-tridecanol, 6-tridecanol, 7-tridecanol, 1-tetradecanol, 2-tetradecanol, 3-tetradecanol, 4-tetradecanol, 5-tetradecanol, 6-tetradecanol, 7-tetradecanol, 1-pentadecanol, 2-pentadecanol, 3-pentadecanol, 4-pentadecanol, 5-pentadecanol, 6-pentadecanol, 7-pentadecanol, 8-pentadecanol, 1-hexadecanol, 2-hexadecanol, 3-hexadecanol, 4-hexadecanol, 5-hexadecanol, 6-hexadecanol, 7-hexadecanol, 8-hexadecanol, 1-heptadecanol, 2-heptadecanol, 3-heptadecanol, 4-heptadecanol, 5-heptadecanol, 6-heptadecanol, 7-heptadecanol, 8-heptadecanol, 1-octadecanol, 2-octadecanol, 3-octadecanol, 4-octadecanol, 5-octadecanol, 6-octadecanol, 7-octadecanol, 8-octadecanol and 9-octadecanol.

Particularly well suited addition products of C₁-C₆ alkylene oxide(s) to aliphatic C₁-C₂₄ alkanols (b5) are the compounds of general formula (AA-I)

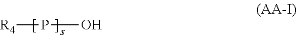

wherein
R4 represents a saturated or unsaturated C₁-C₂₄ alkyl group and
P represents a structural unit —O—CH₂—CH₂—, —O—CH(CH₃)—CH₂— or —O—CH₂—CH(CH₃)—, and
s represents an integer from 1 to 60.

The functional group R4 represents a saturated or unsaturated C₁-C₂₄ alkyl group. The functional groups R4 can be unsaturated from a carbon number of at least 2 C atoms. An unsaturated C₂-C₂₄ alkyl group can comprise one or more double bonds and is alternatively also referred to as an unsaturated C₂-C₂₄ alkenyl group. The saturated or unsaturated C₁-C₂₄ alkyl group can be linear or branched.

Particularly preferably, the functional group R4 is a saturated, unbranched C₁-C₁₂ alkyl group. Very particularly preferably, the functional group R4 is a saturated, unbranched C₁-C₆ alkyl group.

In the compounds of formula (AA-I), P represents a structural unit —O—CH₂—CH₂—, —O—CH(CH₃)—CH₂— or —O—CH₂—CH(CH₃)—. The number of structural units contained in the compounds of formula (AA-I) is obtained by the index number s. In this case, the structural units P are oriented such that the oxygen atom in each group 0-CH₂—CH₂—, —O—CH(CH₃)—CH₂— and —O—CH₂—CH(CH₃)— is adjacent to the alkyl group R4, and the relevant unit —CH₂— or —CH(CH₃)— borders the hydroxy group —OH.

If s is a number greater than 1, a plurality of structural units P are present in the compounds of formula (AA-I). In this case, each structural unit P can be selected independently of the other structural units P.

The index number s represents an integer from 1 to 60, preferably an integer from 1 to 40, further preferably an integer from 10 to 30, and very particularly preferably an integer from 10 to 20.

Within the scope of a further very particularly preferred embodiment, a method according to the invention is characterized in that agent (b) contains:
(b5) at least one addition product of C₁-C₆ alkylene oxide(s) to aliphatic C₁-C₂₄ alkanols of formula (AA-I),

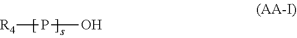

wherein
R4 represents a saturated or unsaturated C₁-C₂₄ alkyl group, preferably a C₁-C₁₂ alkyl group, particularly preferably a C₁-C₆ alkyl group, and
P represents a structural unit —O—CH₂—CH₂—, —O—CH(CH₃)—CH₂— or —O—CH₂—CH(CH₃)—, and
s represents an integer from 1 to 60, preferably an integer from 1 to 40, further preferably an integer from 10 to 30, and very particularly preferably an integer from 10 to 20.

A very particularly well suited addition product of C₁-C₆-alkylene oxide(s) to aliphatic C₁-C₂₄ alkanols of formula (AA-J) is propylene glycol monobutyl ether, which is also referred to as PPG-14 butyl ether and bears the CAS number 9003-13-8. PPG-14 butyl ether can be purchased commercially under the trade name Ucon Fluid AP from Dow.

In a further very particularly preferred embodiment, a method according to the invention is therefore further characterized in that the agent (b) contains PPG-14 butyl ether.

The alkoxylated alkanols (b5) are particularly preferably used within specific quantity ranges in the agent according to the invention.

Particularly good results were obtained when the agent (b) contained, based on the total weight of the agent (b), one or more alkoxylated alkanols (b5) in a total amount of 0.1 to 20.0 wt. %, preferably 0.2 to 15.0 wt. %, more preferably 0.3 to 10.0 wt. %, still more preferably 0.4 to 5.0 wt. %, and very particularly preferably 0.5 to 3.0 wt. %.

Further optional ingredients in agents (a), (b) and (c)

In addition to the already described essential and optional components of the invention, agents (a) and/or (b) and/or (c) can also contain further optional ingredients.

The agents can also contain other active ingredients, auxiliaries and additives, for example surfactants, film-forming polymers, solvents, fatty components, for example of C₈-C₃₀ fatty alcohols, C₈-C₃₀ fatty acid triglycerides, C₈-C₃₀ fatty acid monoglycerides, C₈-C₃₀ fatty acid diglycerides and/or hydrocarbons; polymers; structurants, such as glucose, maleic acid and lactic acid, hair-conditioning compounds, such as phospholipids, for example lecithin and cephalins; perfume oils, dimethyl isosorbide and cyclodextrins; fiber structure-improving agents, in particular mono-, di- and oligosaccharides, for example glucose, galactose, fructose and lactose; dyes for coloring the product; anti-dandruff active ingredients, such as piroctone olamine, zinc omadine and climbazole; amino acids and oligopeptides; animal and/or vegetable-based protein hydrolyzates, as well as in the form of their fatty acid condensation products or optionally anionically or cationically modified derivatives; plant-based oils, light stabilizers and UV blockers; active ingredients, such as panthenol, pantothenic acid, pantolactone, allantoin, pyrrolidinone carboxylic acids and the salts thereof, and bisabolol; polyphenols, in particular hydroxycinnamic acids, 6,7-dihydroxycoumarins, hydroxybenzoic acids, catechins, tannins, leucoanthocyanidins, anthocyanidins, flavanones, flavones and flavonols; ceramides or pseudoceramides; vitamins, provitamins and vitamin precursors; plant extracts; fats and waxes, such as fatty alcohols, beeswax, montan wax and paraffins; swelling agents and penetrants, such as glycerol, propylene glycol monoethyl ether, carbonates, hydrogen carbonates, guanidines, ureas and primary, secondary and tertiary phosphates; opacifiers, such as latex, styrene/PVP and styrene/acrylamide copolymers; pearlescent agents, such as ethylene glycol mono- and distearate as well as PEG-3-distearate; and propellants, such as propane-butane mixtures, N₂O, dimethyl ether, CO₂ and air.

The selection of these additional substances is made by the person skilled in the art according to the desired properties of the agents. With respect to other optional components and the employed amounts of said components, reference is made expressly to relevant manuals known to the person skilled in the art. The additional active ingredients and auxiliaries are used in the preparations according the invention preferably always in amounts of 0.0001 to 25 wt. %, in particular of 0.0005 to 15 wt. %, relative to the total weight of the particular agent.

### Preparation of the Application Mixture by Mixing Agents (a) and (b) and Optionally (c)

In step (4) of the method according to the invention, a ready-to-use mixture is prepared by mixing agents (a) and (b). If a separately packaged pigment is used in a third agent (c), a ready-to-use dye is prepared in step (4) of the method by mixing agents (a) and (b) and (c).

In principle, different amounts of the agent (a) can be mixed with the agent (b); thus, in principle, mixing ratios (a)/(b) of 1:400 to 400:1 are conceivable.

It is particularly preferred if the application mixture is prepared by mixing the agents (a) and (b) in a weight ratio (a)/(b) of from 1:1 to 1:250, preferably from 1:3 to 1:200, more preferably from 1:5 to 1:150, and very particularly preferably from 1:9 to 1:140.
With a quantitative ratio (a)/(b) of 1:130, for example, 1 g of agent (a) can be mixed with 130 g of agent (b).
With a quantitative ratio (a)/(b) of 1:100, for example, 1 g of agent (a) can be mixed with 100 g of agent (b).
With a quantitative ratio (a)/(b) of 1:15, for example, 10 g of agent (a) can be mixed with 150 g of agent (b).
With a quantitative ratio (a)/(b) of 1:25, for example, 4 g of agent (a) can be mixed with 100 g of agent (b).

Within the scope of a further preferred embodiment, a method according to the invention is characterized by
(3) preparing an application mixture by mixing agents (a) and (b) and optionally (c), wherein agents (a) and (b) are mixed with one another in a weight ratio (a)/(b) of 1:1 to 1:250, preferably of 1:3 to 1:200, more preferably of 1:5 to 1:150, and very particularly preferably of 1:9 to 1:140.

### Application of the Application Mixture

In step (5) of the method according to the invention, the application mixture prepared in step (4) is applied to the keratinous material or the keratin fibers or hair.

Preferably, the application mixture is applied to the keratinous material (or to the hair) within a period of 1 to 120 minutes, preferably from 1 to 60 minutes, more preferably from 1 to 30 minutes and very particularly preferably from 1 to 15 minutes after it has been prepared in step (4).

In another preferred embodiment, a method according to the invention is characterized by
(5) applying the application mixture to the keratinous material within a period of 1 to 120 minutes, preferably from 1 to 60 minutes, more preferably from 1 to 30 minutes and very particularly preferably from 1 to 15 minutes after it has been prepared in step (4).

### Exposing the Keratinous Material to the Application Mixture

In step (6) of the method according to the invention, the keratinous material is exposed to the application mixture after the latter has been applied. In this context, various exposure times of, for example, 30 seconds to 60 minutes are conceivable.

However, a great advantage of the dyeing system according to the invention is that an intensive color result can be achieved even in very short periods after short exposure times. For this reason, it is advantageous for the application mixture to remain on the keratinous material after application only for comparatively short periods of 30 seconds to 15 minutes, preferably 30 seconds to 10 minutes, and particularly preferably 1 to 5 minutes.

In another preferred embodiment, a method according to the invention is characterized by
(6) exposure of the keratinous material to the application mixture applied in step (5) for a period from 30 seconds to 15 minutes, preferably from 30 seconds to 10 minutes, and particularly preferably from 1 to 5 minutes.

### Rinsing Out the Application Mixture

Following the exposure of the keratinous material to the application mixture, the latter is lastly rinsed out with water in step (7).

In one embodiment, the application mixture can be washed out with water only, i.e., without the aid of an after-treatment agent or a shampoo. The application of an after-treatment agent or conditioner in a subsequent step (8) is also conceivable in principle.

### Sequence of Method Steps

The method according to the invention comprises steps (1) to (7) (or (1) to (6) if no third agent (c) is to be used).

In step (1), the agent (a) is provided; step (2) comprises providing the agent (b).

In step (3), the optional agent (c) is provided.

These two or three steps do not necessarily have to take place in succession, but can also take place simultaneously.

Step (1) can thus take place before step (2), steps (1) and (2) can take place simultaneously, or step (2) takes place before step (1). Step (3) can take place before or after step (1) or (2). Steps (1), (2) and (3) can also run simultaneously.

If, for example, agents (a) and (b) and optionally (c) are made available to the user in a multi-component packaging unit, the agents are provided simultaneously, and it is left to the user which agent he removes first from the packaging.

It is possible to prepare an application mixture by mixing the agents (a) and (b) and optionally (c) in step (4) only after providing the agents (a) and (b) and optionally (c). It is possible to apply the application mixture in step (5) only after it has been prepared in step (4).

Similarly, it is possible to allow the application mixture to act in step (6) only after it has been applied to the keratinous material, and for the application mixture to be rinsed out in step (7) after it has acted in step (6).

### Multi-Component Packaging Unit

To increase convenience of use, the user is preferably provided with all the required agents in the form of a multi-component packaging unit (kit-of-parts).

A second subject matter of the present invention is therefore a multi-component packaging unit (kit of parts) for treating keratinous material, in particular human hair, comprising, packaged separately from one another:
a first container having an agent (a), wherein agent (a) contains:
(a1) at least one amino-functionalized silicone polymer, and
a second container having an agent (b), wherein agent (b) contains:
(b1) at least one alkylene glycol of formula (AG),

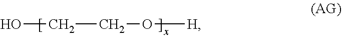

where
x represents an integer from 1 to 10,000, and (b2) at least one solvent from the group consisting of 1,2-propylene glycol, propylene carbonate, 1,3-propylene glycol, 1,2-butylene glycol, dipropylene glycol, ethanol, isopropanol, diethylene glycol monoethyl ether, glycerol, phenoxyethanol and benzyl alcohol, and
optionally a third container having an agent (c), wherein agent (c) contains: (c1) at least one pigment,
wherein agents (a), (b), and (c) and components (a1), (b1), (b2), and (c1) are disclosed in detail in the description of the first subject matter of the invention.

The statements made about the method according to the invention apply mutatis mutandis in regard to the other preferred embodiments of the multi-component packaging unit according to the invention.

### EXAMPLES

### 1. Formulations

The following formulations were produced:

| Agent (a) | (a) |
|---|---|
| Dow Corning 2-8566 (siloxanes and silicones, | 0.75 g |
| 3-[(2-aminoethyl)amino]-2-methylpropyl Me, | |
| Di-Me-siloxane) | |
| Total amount of agent (a) | 0.75 g |

| | (bV) | | (bE) | |
|---|---|---|---|---|
| Agent (b) | Comparison | | Invention | |
| Water | 10.0 | g | 10.0 | g |
| Plantacare 2000 UP (DECYL | 0.38 | g | 0.38 | g |
| GLUCOSIDE, water) | | | | |
| PPG-3 benzyl ether myristate | 1.5 | g | 1.5 | g |
| Ucon Fluid AP (PPG-14 Butyl Ether) | 0.4 | g | 0.4 | g |
| 4-hydroxyacetophenone | 0.2 | g | 0.2 | g |
| Phenoxyethanol | 0.9 | g | 0.9 | g |
| 1,2-propanediol | — | | 30.0 | g |
| Unipure Red LC 3079 (Pigment) | 1.0 | g | 1.0 | g |
| Polyethylene glycol (molecular | 10.0 | g | 10.0 | g |
| weight 6000 g/mol) | | | | |
| Polyethylene glycol (molecular | ad 100 | g | ad 100 | g |
| weight 400 g/mol) | | | | |
| Total amount of agent (b) | 100 | g | 100 | g |

| Agent (c) | (c) |
|---|---|
| Silver pigment (Alegrace Marvelous | 1.0 g |
| D 12/77-1 Shiny Silver, 10% | |
| aqueous dispersion) | |
| Total amount of agent (c) | 1 g |

### 2. Preparation of the Application Mixture by Mixing (a) and (b)

The agent (b) was filled into an applicator bottle in each case. The agent (a) was then added, and the agents (a) and (b) were stirred together with the aid of a spatula. Using a gloved hand, 4 g of the application mixture from (a) and (b) was applied to a strand of hair (Kerling, type “Euronatur-haar weiß”, 4 g), massaged in for 10 seconds and allowed to act for 5 minutes. The strands were then rinsed with water and dried. After drying, the dyed strand was fanned apart and the homogeneity of the color result was visually assessed by a trained person.

| AWM 1, comparison | AWM 2, invention |
|---|---|
| 0.75 g agent (a) | 0.75 g agent (a) |
| 100 g agent (bV) | 100 g agent (bE) |
| non-uniform color result; | uniform color result |
| in particular the inner | in the entire strand |
| section of the strand is | section |
| more weakly dyed | |

### 3. Preparation of the Application Mixture by Mixing (a) and (b) and (c)

The agent (b) was filled into an applicator bottle. Agent (a) and agent (c) were then added, and the three agents (a), (b) and (c) were stirred together with the aid of a spatula. Using the gloved hand, 4 g of the application mixture from (a), (b) and (c) was applied to a strand of hair (Kerling, type “Euronatur-haar weiß”, 4 g), massaged in for 10 seconds and allowed to act for 5 minutes. The strands were then rinsed with water and dried. After drying, the dyed strand was fanned apart and the homogeneity of the color result was visually assessed by a trained person.

| AWM 3, comparison | AWM 4, invention |
|---|---|
| 0.75 g agent (a) | 0.75 g agent (a) |
| 100 g agent (bV) | 100 g agent (bE) |
| 1.0 g agent (c) | 1.0 g agent (c) |
| non-uniform color result; | uniform color result |
| in particular the inner | in the entire strand |
| section of the strand is | section, uniform |
| more weakly dyed, | silver shimmer |
| patchy silver shimmer | |

What is claimed is:

1. A method for treating keratinous material, comprising:
(1) providing an agent (a), the agent (a) comprising:
(a1) at least one amino-functionalized silicone polymer, and
(2) providing an agent (b), the agent (b) containing:
(b1) at least one alkylene glycol of formula (AG), 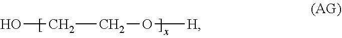
where
x represents an integer from 1 to 10,000, and
(b2) at least one solvent selected from the group consisting of 1,2-propylene glycol, propylene carbonate, 1,3-propylene glycol, 1,2-butylene glycol, dipropylene glycol, ethanol, isopropanol, diethylene glycol monoethyl ether, glycerol, phenoxyethanol and benzyl alcohol, and
(3) optionally providing an agent (c), the agent (c) comprising:
(c1) at least one pigment,
(4) preparing an application mixture by mixing agents (a) and (b) and optionally (c),
(5) applying the application mixture prepared in step (4) to the keratinous material,
(6) exposing the keratinous material to the application mixture applied in step (5), and
(7) optionally rinsing out the application mixture.

2. The method according to claim 1, wherein the at least one amino-functionalized silicone polymer (a1) comprises at least one structural unit of the formula (Si-amino): 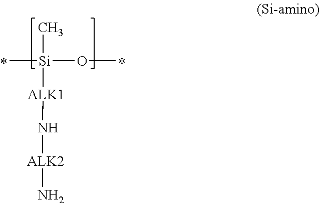
where
ALK1 and ALK2 represent, independently of one another, a linear or branched divalent C₁-C₂₀ alkylene group.

3. The method according to claim 1, wherein the at least one amino-functionalized silicone polymer (a1) comprises structural units of the formula (Si—I) and of the formula (Si-II)

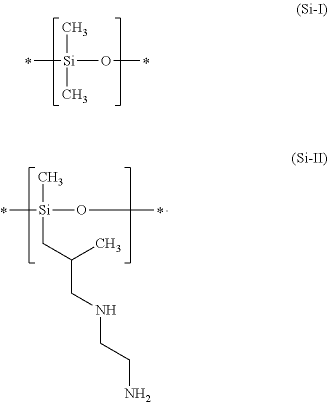

4. The method according to one of claim 1, wherein the agent (a) contains, based on the total weight of the agent (a), one or more amino-functionalized silicone polymers (a1) in a total amount from 1 to 100 wt. %.

5. The method according to claim 1, wherein the agent (b) contains (b1) at least one alkylene glycol of the formula (AG-1), 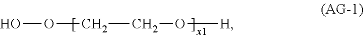
where x1 is an integer from 1 to 100.

6. The method according to claim 1, wherein the agent (b) contains (b1) at least one alkylene glycol of the formula (AG-2), 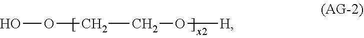
where x2 denotes an integer from 101 to 1000.

7. The method according to claim 1, wherein the agent (b) contains, based upon the total weight of the agent (b), one or more alkylene glycols (b1) of formula (AG) in a total amount of 10.0 to 99.0 wt. %.

8. The method according to claim 1, wherein the agent (b) contains, based on the total weight of the agent (b), one or more solvents (b2) in a total amount from 1 to 80 wt. %.

9. The method according to claim 1, wherein the agent (b) comprises:
(b3) at least one pigment.

10. The method according to claim 1, further comprising the steps of:
(3) providing an agent (c), the agent (c) containing:
(c1) at least one pigment, and
(4) preparing an application mixture by mixing agents (a) and (b) and (c).

11. The method according to claim 1, wherein the agent (b) comprises:
(b4) at least one alkoxylated fatty acid ester of general formula (AFE-I) 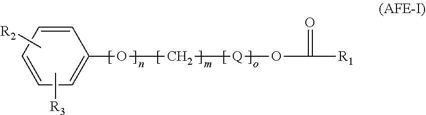
where
R1 represents a saturated or unsaturated C₁₁-C₂₉ alkyl group,
R1, R3 represent, independently of one another, a hydrogen atom, a C₁-C₆ alkyl group, a hydroxy group, or a C₁-C₆ alkoxy group,
n represents the number 0 or 1,
m represents an integer from 0 to 6,
o represents an integer from 1 to 60, and
Q represents a structural unit —O—CH₂—CH₂—, —O—CH(CH₃)—CH₂— or —O—CH₂—CH(CH₃)—
with the proviso that if m is equal to 0, n is also equal to 0.

12. The method according to claim 1, wherein the agent (b) comprises:
(b5) at least one addition product of C₁-C₆ alkylene oxide(s) to aliphatic C₁-C₂₄ alkanols of formula (AA-I), 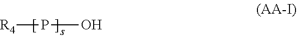
wherein
R4 represents a saturated or unsaturated C₁-C₂₄ alkyl group, and
P represents a structural unit —O—CH₂—CH₂—, —O—CH(CH₃)—CH₂— or —O—CH₂—CH(CH₃)—, and
s represents an integer from 1 to 60.

13. The method according to claim 1, wherein agents (a) and (b) are mixed with one another in a weight ratio (a)/(b) of 1:1 to 1:250.

14. A multi-component packaging unit (kit-of-parts) for treating keratinous material, comprising, packaged separately from one another,
a first container having an agent (a), wherein the agent (a) contains:
(a1) at least one amino-functionalized silicone polymer, and
a second container having an agent (b), wherein the agent (b) contains:
(b1) at least one alkylene glycol of formula (AG) 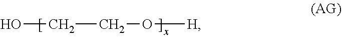
where
x represents an integer from 1 to 10,000, and
(b2) at least one solvent selected from the group consisting of 1,2-propylene glycol, propylene carbonate, 1,3-propylene glycol, 1,2-butylene glycol, dipropylene glycol, ethanol, isopropanol, diethylene glycol monoethyl ether, glycerol, phenoxyethanol and benzyl alcohol, and
optionally a third container having an agent (c), wherein the agent (c) contains:
(c1) at least one pigment.
